(19) United States
(12) Patent Application Publication
(10) Pub. No.: US 2023/0029387 A1
(43) Pub. Date: Jan. 26, 2023

(54) EXPANDABLE SHEATH FOR A TRANSCATHETER HEART VALVE

(71) Applicant: MIRUS LLC, Marietta, GA (US)

(72) Inventors: Jay Yadav, Marietta, GA (US); Angela Rothfuss, Marietta, GA (US); Darren DeMedici, Marietta, GA (US); Noah Roth, Marietta, GA (US)

(21) Appl. No.: 17/873,808

(22) Filed: Jul. 26, 2022

Related U.S. Application Data
(60) Provisional application No. 63/225,803, filed on Jul. 26, 2021.
(60) Provisional application No. 63/388,782, filed on Jul. 13, 2022.

(51) Int. Cl. A61M 25/00; A61F 2/24

(52) U.S. Cl. CPC A61M 25/0023; A61F 2/2466; A61M 2025/0024; A61M 2205/0266

(57) ABSTRACT

The present disclosure relates to a sheath that is usable with a medical device, particularly to a sheath that is useable with a medical device and which sheath is expandable in the regions that the medical device is positioned in the sheath, and more particularly to a sheath that is useable with a medical device and which sheath is expandable in the regions that the medical device is positioned in the sheath and which sheath reforms to its same or similar size and shape after the medical device has passed through a portion of all of the sheath. The sheath is used to protect the body passageway of a patient as a medical device is inserted into and/or through the body passageway and to a treatment site.

The present disclosure claims priority on U.S. Provisional Application Ser. No. 63/225,803 filed Jul. 26, 2021, which is fully incorporated herein by reference.

The present disclosure claims priority on U.S. Provisional Application Ser. No. 63/388,782 filed Jul. 13, 2022, which is fully incorporated herein by reference.

The present disclosure relates generally to a sheath that is usable with a medical device, particularly to a sheath that is useable with a medical device and which sheath is expandable in at least the regions that the medical device is positioned in the expandable sheath, and more particularly to a sheath that is useable with a medical device and which sheath is expandable in at least the regions that the medical device is positioned in the expandable sheath and which sheath reforms to its same or similar size and shape after the medical device has passed through a portion of all of the expandable sheath. The expandable sheath is can be used to protect the body passageway of a patient as a medical device is inserted into and/or through the body passageway and to a treatment site. The expandable sheath that can be used with catheter arrangements for repairing and/or replacing heart valves.

### BACKGROUND

Many modern-day medical devices such as stents and heart valves are inserted through blood vessels until the medical device reaches a treatment site, and thereafter the medical device is expanded at the treatment site to secure the medical device at the treatment site. Many medical devices are formed or include metal materials that can include sharp or abrasive edges or ends. Also, the size of the medical device as is passes through a body passageway can cause trauma to the body passageway as the medical device and delivery device are inserted through the body passageway to the treatment site and when the delivery device is removed from the body passageway after delivery of the medical device at the treatment site.

Endovascular delivery catheter assemblies are used to implant prosthetic devices, such as a prosthetic valve, at locations inside the body that are not readily accessible by surgery or where access without invasive surgery is desirable. For example, aortic, mitral, tricuspid, and/or pulmonary prosthetic valves can be delivered to a treatment site using minimally invasive surgical techniques.

A sheath can be used to introduce a delivery apparatus into a patient's vasculature. A sheath can be used to minimize damage to a body passageway as the delivery apparatus is inserted into a body passageway (e.g., the femoral artery, etc.). A sheath generally has an elongated sleeve that is inserted into the vasculature and a housing that allows a delivery apparatus to be placed in fluid communication with the vasculature with minimal blood loss. A conventional sheath typically requires a tubular loader to be inserted through the seals in the housing to provide an unobstructed path through the housing for a valve mounted on a balloon catheter. A conventional loader extends from the proximal end of the expandable sheath. One prior art sheath is disclosed in U.S. Pat. No. 11,045,317, which is fully incorporated herein by reference.

Conventional methods of accessing a vessel prior to introducing the delivery system include dilating the vessel using multiple dilators or sheaths that progressively increase in diameter.

Radially expanding intravascular sheaths tend to have complex mechanisms, such as ratcheting mechanisms that maintain the shaft or sheath in an expanded configuration once a device with a larger diameter than the expandable sheath's original diameter is introduced.

The delivery and/or removal of medical device to or from the body passageway of a patient can expose the patient to risk. Furthermore, accessing a body passageway and inserting the medical device into a body passageway with damaging the medical device and/or severely damaging the body passageway continues to be a challenge due in part to the relatively large profile of the delivery system that can cause longitudinal and radial tearing of the vessel during insertion.

Accordingly, there remains a need for an improved sheath for endovascular systems used for implanting valves, and other medical devices.

### SUMMARY OF THE DISCLOSURE

The present disclosure relates generally to a sheath that is usable with a medical device, particularly to a sheath that is useable with a medical device and which sheath is expandable in at least the regions that the medical device is positioned in the expandable sheath, and more particularly to an expandable sheath that is useable with a medical device and which expandable sheath is expandable in at least the regions that the medical device is positioned in the expandable sheath and which sheath reforms to its same or similar size and shape after the medical device has passed through a portion of all of the expandable sheath. The expandable sheath is can be used to protect the body passageway of a patient as a medical device is inserted into and/or through the body passageway and to a treatment site. The expandable sheath that can be used with catheter arrangements for repairing and/or replacing heart valves.

In one non-limiting aspect of the present disclosure, the expandable sheath can be used to reduce or minimize trauma to a body passageway (e.g., blood vessel, duct, etc.) the vessel by allowing for temporary expansion of a portion of the expandable sheath to accommodate at least a portion of the delivery system and/or medical device, followed by a return to the same or similar original diameter once the delivery system and/or medical device has partially or fully passes through the expandable sheath. In one non-limiting embodiment, the expandable sheath is configured to expand from a first outer diameter to a second outer diameter as a medical device (e.g., TAVR, stent, etc.) is moved through an interior passageway in the expandable sheath, and thereafter the expandable sheath partially or fully returns to the first outer diameter once the medical device has passed partially or fully through the expandable sheath. In another non-limiting embodiment, the expandable sheath can be configured such that the outer diameter or cross-sectional area of the expandable sheath and the diameter or cross-sectional area of the interior passageway progressively expands and contracts as the medical device is moved through the expandable sheath. In such a configuration, only a portion of the expandable sheath where the medical device is located in the interior passageway of the expandable sheath expands to from the first to the second outer diameter. In the region of the expandable sheath that is located forwardly and/or rearwardly of the when the medical device is located in the expandable sheath, the expandable sheath may have an outer diameter that is the same or similar to the non-expanded first diameter or have an outer diameter that is greater than the non-expanded first diameter, but is less than the outer diameter of the expandable sheath where in the medical device is located in the expandable sheath. As such, the expandable sheath can be configured to progressively increase in outer diameter and then decrease in outer diameter as the medical device moves through the interior passageway of the expandable sheath. In another non-limiting embodiment, the expandable sheath can be configured such that the outer diameter or cross-sectional area of the expandable sheath and the diameter or cross-sectional area of the interior passageway expands to a second diameter along the longitudinal length of the expandable sheath as the medical device moves through the expandable sheath, and then partially or fully returns to the first outer diameter once the medical device has fully passes through the expandable sheath. In another non-limiting embodiment, the expandable sheath is partially for fully formed of a shape memory material that is used to facilitate in causing the expandable sheath to partially or fully return to the first outer diameter once the medical device has partially or fully passed through the expandable sheath.

In another and/or alternative non-limiting aspect of the present disclosure, the expandable sheath provides access to the vasculature for delivery of medical devices or for medical intervention, and wherein the expandable sheath includes: a) a plurality of bands, each of the bands including first and second ends; and b) an outer layer that cases one or more of the bands; and wherein the band and/or the outer layer are formed of a polymer and/or a metal material; and wherein the first end of each of the bands is bonded to, connected to or merges with an adjacently positioned band; and wherein the second end of each of the bands is bonded to, connected to or merges with an adjacently positioned band; and wherein the outer layer is bonded to, connected to or merges with one or more of the bands; and wherein the expandable sheath is expandable between a fully contracted orientation and a fully expanded orientation.

In another and/or alternative non-limiting aspect of the present disclosure, the expandable sheath for delivering a medical device (e.g., prosthetic heart valve, stent, graft, etc.), the expandable sheath comprising: a) a plurality of thin bands, each of the thin bands including first and second ends; and b) a plurality of thick bands, each of the thick band including first and second ends; and wherein the thin band and/or the thick bands are partially or fully formed of a polymer and/or a metal material; and wherein the first end of each of the thin band is connected to or merges with the second end of an adjacently positioned thick band, and the second end of each of the thin bands is connected to or merges with the first end of an adjacently positioned thick band; and wherein the expandable sheath is expandable between a first contracted orientation and a second expanded orientation.

In another and/or alternative non-limiting aspect of the present disclosure, the expandable sheath for introducing a medical device (e.g., prosthetic heart valve, stent, graft, etc.) is configured to locally expand from a first diameter to a second diameter as the medical device is moved through a lumen of the expandable sheath, and then at least partially returned to the first diameter or some diameter between the first and second diameter once the medical device has partially or fully passed through the expandable sheath.

In another and/or alternative non-limiting aspect of the present disclosure, the expandable sheath is partially or fully formed of a polymer material. Non-limiting polymers that can be used include polytetrafluoroethylene (PTFE), polyimide, polyetheretherketone (PEEK), polyurethane, nylon, polyethylene, polyamide, styrenic block copolymer (SBC) (e.g., polystyrene blocks and rubber blocks [e.g., polybutadiene, polyisoprene or their hydrogenated equivalents]), polyether block amides, polyether block ester copolymer, thermoset silicone, latex, poly-isoprene rubbers, high density polyethylene (HDPE), polyurethane resin, elastomers formed of block copolymers made up of rigid polyamide blocks and soft polyether blocks, or combinations thereof. In one non-limiting embodiment, the expandable sheath is formed of 60-100 wt. % (and all values and ranges therebetween) of polymer. In one non-limiting specific configuration, 100 wt.% of the expandable sheath is formed of one type of polymer (e.g., styrenic block copolymer (SBC)). In another non-limiting specific configuration, 10-49 wt. % of the expandable sheath is formed of one type of polymer (e.g., styrenic block copolymer (SBC), etc.), and 51-90 wt. % of the expandable sheath is formed of another polymer (e.g., high density polyethylene (HDPE), etc.). In one non-limiting embodiment, the expandable sheath includes a shape memory material. The shape member material is or includes a) copper-aluminum-nickel alloy, b) nickel-titanium alloy, c) zinc-copper-gold-iron alloy, d) Fe—Mn—Si alloy, e) Cu—Zn—Al alloy, f) Cu—Al—Ni alloy, g) Ag—Cd alloy, h) Au—Cd alloy, i) Co—Ni—Al alloy, j) Co—Ni—Ga alloy, k) Cu—Al—Be—X (X═Zr, B, Cr, Gd) alloy, l) Cu—Al—Ni—Hf alloy, m) Cu—Sn alloy, n) Cu—Zn alloy, o) Cu—Zn—X (X═Si, Al, Sn) alloy, p) Fe—Pt alloy, q) Mn—Cu alloy, r) Ni—Fe—Ga alloy, s) Ni—Ti—Hf alloy, t) Ni—Ti—Pd alloy, u) Ni—Mn—Ga alloy, v) Ni—Mn—Ga—Cu alloy, w) Ni—Mn—Ga—Co alloy, x) Ti—Nb alloy, y) polyacrylate-based SMPs (e.g., t-butylacrylate-co-poly(ethyleneglycol) dimethacrylate (tBA-co-PEGDMA) polymers, etc.), z) (meth)acrylate-based SMPs, aa) polyurethane-based SMPs, and/or bb) blends of polyurethane and polyvinylchloride-based SMPs. In one non-limiting arrangement, the expandable sheath includes a shape memory polymer material. In another non-limiting arrangement, the expandable sheath includes a shape memory metal.

In another and/or alternative non-limiting aspect of the present disclosure, the expandable sheath is formed of multiple materials. For example, one or more portions of the expandable sheath can be formed to be rigid and one or more other portions of the expandable sheath can be formed to be more flexible. The materials used to form the rigid portions and the flexible portions can be the same or different. In one non-limiting embodiment, the cross-sectional area and/or thickness of the rigid portions are greater than the cross-sectional area and/or thickness of the flexible portions; however, this is not required. In another non-limiting embodiment, the total weight percent and/or volume percent of the rigid portions of the expandable sheath is greater than the total weight percent and/or volume percent of the flexible portions of the expandable sheath; however, this is not required.

In another and/or alternative non-limiting aspect of the present disclosure, the expandable sheath includes a plurality of rigid portions that are connected at one or both ends by the more flexible portions. In one specific non-limiting configuration, the expandable sheath includes a plurality of rigid portions that are all connected at their ends to the ends to a more flexible portion. In another specific non-limiting configuration, the expandable sheath includes a plurality of rigid portions and a plurality of more flexible portions that are formed of the same type of material (e.g., high density polyethylene (HDPE)). In another specific non-limiting configuration, the expandable sheath includes a plurality of rigid portions that are formed of one type of material (e.g., high density polyethylene (HDPE)) and a plurality of flexible portions that are formed of another type of material (e.g., high density polyethylene (HDPE)).

In another and/or alternative non-limiting aspect of the present disclosure, the expandable sheath is expandable between a contracted orientation to an expanded orientation and had a generally circular cross-sectional profile in both the contracted orientation and the expanded orientation. The diameter of the expandable sheath in the expanded orientation is greater than the diameter of the expandable sheath in the contracted orientation.

In another and/or alternative non-limiting aspect of the present disclosure, the expandable sheath in the contracted orientation has a cross-sectional wall thickness that is generally the same (±0-5% and all values and ranges therebetween) for 70%-98% (and all values and ranges therebetween) of the circumference of the expandable sheath in the contracted orientation, and the expandable sheath in the expanded orientation has a cross-sectional wall thickness that is generally the same (±0-5% and all values and ranges therebetween) for 0-65% (and all values and ranges therebetween) of the circumference of the expandable sheath in the expanded orientation.

In another and/or alternative non-limiting aspect of the present disclosure, the expandable sheath can optionally include a hemostasis valve at or near one end of the expandable sheath.

In another and/or alternative non-limiting aspect of the present disclosure, the expandable sheath has a constant cross-sectional size and shape along 50-100% (and all values and ranges therebetween) of the longitudinal length of the expandable sheath when the expandable sheath is in the contracted orientation and/or when the expandable sheath is in the expanded orientation. In another non-limiting embodiment, the outer surface of the expandable sheath and/or the interior surface of the expandable sheath has a constant size and shape along 50-100% (and all values and ranges therebetween) of the longitudinal length of the expandable sheath when the expandable sheath is in the contracted orientation and/or when the expandable sheath is in the expanded orientation.

In another and/or alternative non-limiting aspect of the present disclosure, the interior surface (i.e., the surface of the interior cavity of the expandable sheath) and/or exterior surface of the expandable sheath can optionally be coated with one or more polymers, drugs, etc.).

In another and/or alternative non-limiting aspect of the present disclosure, the expandable sheath includes a plurality of segments that have overlapping portions that are each configured to locally partially or fully move to a non-overlapping orientation, thus increasing the length of such segments, when the expandable sheath is expanded from the contracted orientation to the expanded orientation. One or more of these segments can optionally be configured to at least partially return to the overlapping orientation when an interior force on the interior cavity of the expandable sheath is reduced (e.g., deflation of an expandable balloon, a medical device has partially or fully passed through the expandable sheath, etc.).

In another and/or alternative non-limiting aspect of the present disclosure, the expandable sheath can be formed by an extrusion process, stamping process or casting process. In one non-limiting embodiment, the expandable sheath is formed by an extrusion process.

In another and/or alternative non-limiting aspect of the present disclosure, the expandable sheath includes multiple layers. In non-limiting embodiment, the expandable sheath includes an outer layer portion, a frame and an inner layer portion. One non-limiting arrangement the outer layer portion, the frame and the inner layer portion facilitate in maintaining the shape and integrity of the expandable sheath.

In another and/or alternative non-limiting aspect of the present disclosure, the expandable sheath includes an inner layer portion that can be formed of one or more polymer layers. In one non-limiting embodiment, the one or more polymer layers used to partially or fully form the inner layer portion can include polyethylene, polytetrafluoroethylene, polyimide, polyetheretherketone, polyurethane, nylon, polyether block amides, polyether block ester copolymer, thermoset silicone, latex, poly-isoprene rubbers, styrene ethylene butylene styrene, polyesters, fluoropolymers, polyvinyl chloride, polyolefin, and/or high-density polyethylene. In one non-limiting configuration, the inner layer portion is or includes polyethylene. In another non-limiting embodiment, the inner layer portion is formed of a single polymer layer. In another non-limiting embodiment, the inner layer portion is formed of at least two polymer layers. When the inner layer portion includes two or more layers, the composition of the layers can be the same or different. In one non-limiting configuration, the inner layer portion is formed of two layers, and wherein the inner layer is formed of high density polyethylene and the outer layer is formed of styrene ethylene butylene styrene, and wherein the two layers are optionally co-extruded, and wherein the inner layer forms 30-70% (and all values and ranges therebetween of the total thickness of the inner layer portion and the outer layer forms 30-70% (and all values and ranges therebetween of the total thickness of the inner layer portion. In another non-limiting embodiment, the thickness of the inner layer portion is generally at least 0.002 inches, typically 0.002-0.02 inches (and all values and ranges therebetween), and more typically 0.004-0.008 inches. In another non-limiting embodiment, the inner layer portion is formed of a material that allows the outer diameter or outer cross-sectional area of the expandable sheath and the diameter or cross-sectional area of the interior passageway to expand and contract as the medical device is moved through the expandable sheath without damaging the material that forms inner layer portion. In another non-limiting embodiment, when the inner layer portion is formed of two or more layers, the inner layer portion can optionally be formed by a co-extrusion process.

In another and/or alternative non-limiting aspect of the present disclosure, the expandable sheath includes an outer layer portion that can be formed of one or more polymer layers. The outer layer portion can be formed of the same or similar material as the inner layer portion. In one non-limiting embodiment, the one or more polymer layers used to partially or fully form the outer layer portion can include polyethylene, polytetrafluoroethylene, polyimide, polyetheretherketone, polyurethane, nylon, polyether block amides, polyether block ester copolymer, thermoset silicone, latex, poly-isoprene rubbers, styrene ethylene butylene styrene, polyesters, fluoropolymers, polyvinyl chloride, polyolefin, and/or high-density polyethylene. In one non-limiting configuration, the outer layer portion is or includes polyethylene. In another non-limiting embodiment, the outer layer portion is formed of a single polymer layer. In another non-limiting embodiment, the outer layer portion is formed of at least two polymer layers. When the outer layer portion includes two or more layers, the composition of the layers can be the same or different. In one non-limiting configuration, the outer layer portion is formed of two layers, and wherein the outer layer is formed of high density polyethylene and the inner layer is formed of styrene ethylene butylene styrene, and wherein the two layers are optionally co-extruded, and wherein the inner layer forms 30-70% (and all values and ranges therebetween of the total thickness of the outer layer portion and the outer layer forms 30-70% (and all values and ranges therebetween of the total thickness of the outer layer portion. In another non-limiting embodiment, the thickness of the outer layer portion is generally at least 0.002 inches, typically 0.002-0.03 inches (and all values and ranges therebetween), and more typically 0.004-0.015 inches. In another non-limiting embodiment, the thickness of the outer layer portion is greater than the thickness of the inner layer portion. In another non-limiting embodiment, the outer layer portion is formed of a material that allows the outer diameter or outer cross-sectional area of the expandable sheath and the diameter or cross-sectional area of the interior passageway to expand and contract as the medical device is moved through the expandable sheath without damaging the material that forms outer layer portion. In another non-limiting embodiment, when the outer layer portion is formed of two or more layers, the outer layer portion can optionally be formed by a co-extrusion process.

In another and/or alternative non-limiting aspect of the present disclosure, the expandable sheath includes a frame includes a shape memory material. In one non-limiting embodiment, the majority (e.g., 60-99.99% and all values and ranges therebetween) or all of the frame is formed of a shape member material. In another non-limiting embodiment, the shape member material is or includes a) copper-aluminum-nickel alloy, b) nickel-titanium alloy, c) zinc-copper-gold-iron alloy, d) Fe—Mn—Si alloy, e) Cu—Zn—Al alloy, f) Cu—Al—Ni alloy, g) Ag—Cd alloy, h) Au—Cd alloy, i) Co—Ni—Al alloy, j) Co—Ni—Ga alloy, k) Cu—Al—Be—X (X═Zr, B, Cr, Gd) alloy, 1) Cu—Al—Ni—Hf alloy, m) Cu—Sn alloy, n) Cu—Zn alloy, o) Cu—Zn—X (X═Si, Al, Sn) alloy, p) Fe—Pt alloy, q) Mn—Cu alloy, r) Ni—Fe—Ga alloy, s) Ni—Ti—Hf alloy, t) Ni—Ti—Pd alloy, u) Ni—Mn—Ga alloy, v) Ni—Mn—Ga—Cu alloy, w) Ni—Mn—Ga—Co alloy, x) Ti—Nb alloy, y) polyacrylate-based SMPs (e.g., t-butylacrylate-co-poly(ethyleneglycol) dimethacrylate (tBA-co-PEGDMA) polymers, etc.), z) (meth)acrylate-based SMPs, aa) polyurethane-based SMPs, and/or bb) blends of polyurethane and polyvinylchloride-based SMPs. In one non-limiting configuration, the frame is partially or fully formed of nickel-titanium alloy.

In another and/or alternative non-limiting aspect of the present disclosure, the thickness of the frame of the expandable sheath is generally at least 0.002 inches, typically 0.002-0.03 inches (and all values and ranges therebetween), and more typically 0.004-0.012 inches. In another non-limiting embodiment, the thickness of the frame is greater than the thickness of the inner layer portion. In another non-limiting embodiment, the thickness of the frame is equal to or less than the thickness of the outer layer portion.

In another and/or alternative non-limiting aspect of the present disclosure, the frame of the expandable sheath is formed of a material that allows the outer diameter or outer cross-sectional area of the expandable sheath and the diameter or cross-sectional area of the interior passageway to expand and contract as the medical device is moved through the expandable sheath without damaging the frame.

In another and/or alternative non-limiting aspect of the present disclosure, the frame of the expandable sheath has a configuration that allows the outer diameter or outer cross-sectional area of the expandable sheath and the diameter or cross-sectional area of the interior passageway to expand and contract as the medical device is moved through the expandable sheath without damaging the frame. In one non-limiting embodiment, the frame provides strength, structure and/or shape to the expandable sheath. In another non-limiting embodiment, the frame partially or fully includes a non-overlapping structure (e.g., few or no interlocking fingers or struts, etc.). In another non-limiting embodiment, the frame includes two sets of undulating wires along the longitudinal length of the expandable sheath. In one non-limiting configuration, the undulating wires, when used, are configured to not overlap one another along the partial or full longitudinal length of the expandable sheath. In another non-limiting configuration, the undulating wires, when used, one or more undulating wires of the frame have a generally sinusoidal shape. In another non-limiting configuration, the frame includes two or more undulating wires, and wherein at least two of the wires have the same shape; however, this is not required. In another non-limiting embodiment, the pattern and shape of the wires of the frame enables the outer diameter or outer cross-sectional area of the expandable sheath and the diameter or cross-sectional area of the interior passageway to expand and contract as the medical device is moved through the expandable sheath without damaging the frame. In another non-limiting embodiment, the shape memory material used to at least partially form the frame causes the expandable sheath to contact to its original or near original shape after being expanded as the medical device passes through the expandable sheath. In another non-limiting embodiment, the material used to form each of the wires of the frame is the same; however, this is not required. In another non-limiting embodiment, the shape and size of each of the wires of the frame are the same; however, this is not required.

In another and/or alternative non-limiting aspect of the present disclosure, the expandable sheath optionally includes one or more longitudinal shaping structures positioned along the longitudinal length of the expandable sheath. The one or more longitudinal shaping structures are configured to inhibit or prevent kinking of the expandable sheath during use and during insertion and removal of the expandable sheath from a body passageway. The one or more longitudinal shaping structures can also or alternatively be used to inhibit or prevent the compressing and/or elongation of the expandable sheath while it a) expands in diameter, b) reduces in diameter, c) is inserted into a body passageway, or d) is removed from the body passageway. In one non-limiting embodiment, the one or more longitudinal shaping structures can be in the form of one or more wires that extend partially or fully along the longitudinal length of the expandable sheath. When two of more wires are used, the wires are generally spaced from one another. In another non-limiting embodiment, the one or more longitudinal shaping structures can extend along 50-100% (and all values and ranges therebetween) the longitudinal length of the expandable sheath. In another non-limiting embodiment, the spacing of two or more of the longitudinal shaping structures can be about a 10-50% (and all values and ranges therebetween) of the outer circumference of the expandable sheath from one another. In another non-limiting embodiment, a large gap or spacing can optionally be formed between two of the longitudinal shaping structures. The large gap can about 30-80% (and all values and ranges therebetween) of the outer circumference of the expandable sheath. The large gap or spacing exists, when used, can facilitates in the bending of the expandable sheath in the location absent the longitudinal shaping structures. In another non-limiting embodiment, the one or more longitudinal shaping structures are partially or fully formed of a shape memory material. The shape memory material used to partially or fully form the one or more longitudinal shaping structures can be used to cause the expandable sheath to straighten along its longitudinal axis, thereby inhibiting or preventing kinking of the expandable sheath when inserted and/or removed from a body passageway. The composition of shape memory material can be a) copper-aluminum-nickel alloy, b) nickel-titanium alloy, c) zinc-copper-gold-iron alloy, d) Fe—Mn—Si alloy, e) Cu—Zn—Al alloy, f) Cu—Al—Ni alloy, g) Ag—Cd alloy, h) Au—Cd alloy, i) Co—Ni—Al alloy, j) Co—Ni—Ga alloy, k) Cu—Al—Be—X (X═Zr, B, Cr, Gd) alloy, l) Cu—Al—Ni—Hf alloy, m) Cu—Sn alloy, n) Cu—Zn alloy, o) Cu—Zn—X (X═Si, Al, Sn) alloy, p) Fe—Pt alloy, q) Mn—Cu alloy, r) Ni—Fe—Ga alloy, s) Ni—Ti—Hf alloy, t) Ni—Ti—Pd alloy, u) Ni—Mn—Ga alloy, v) Ni—Mn—Ga—Cu alloy, w) Ni—Mn—Ga—Co alloy, x) Ti—Nb alloy, y) polyacrylate-based SMPs (e.g., t-butylacrylate-co-poly(ethyleneglycol) dimethacrylate (tBA-co-PEGDMA) polymers, etc.), z) (meth)acrylate-based SMPs, aa) polyurethane-based SMPs, and/or bb) blends of polyurethane and polyvinylchloride-based SMPs. In one non-limiting configuration, the one or more longitudinal shaping structures are partially or fully formed of a nickel-titanium alloy. In another non-limiting embodiment, the one or more longitudinal shaping structures are positioned in the inner and/or outer layer portions of the expandable sheath. In one non-limiting configuration, the one or more longitudinal shaping structures are encapsulated in the inner and/or outer layer portions of the expandable sheath. In one non-limiting configuration, the one or more longitudinal shaping structures are encapsulated between the inner and outer layers of the inner and/or outer layer portions of the expandable sheath; however, this is not required. In one non-limiting configuration, the one or more longitudinal shaping structures are coextruded with the inner and/or outer layer portions; however, other processes can be used to secure the one or more longitudinal shaping structures to and/or in the inner and/or outer layer portions (e.g., adhesive, mechanical connection, etc.). In another non-limiting configuration, after the one or more longitudinal shaping structures are positioned between layers of the inner and/or outer layer portions, the inner and/or outer layer portions is optionally reflowed and/or the layers are optionally adhesively connected together to secure together the layers of the inner and/or outer layer portions. Such securing together of the layers facilitates in maintaining the one or more longitudinal shaping structures in position in the expandable sheath. When the one or more longitudinal shaping structures are in the form of a wire, the size and shape of the wire can be the same or similar to the size and shape of the wire used to form the frame; however, this is not required. In another non-limiting embodiment, the one or more longitudinal shaping structures are spaced from the frame and/or spaced from the inner layer portion.

In another and/or alternative non-limiting aspect of the present disclosure, one or both ends of the expandable sheath are subjected to a connection processed so as to connect together the outer layer portion and the inner layer portion. One non-limiting connection process is a reflow process to secure the outer layer portion and the inner layer portion by a heat bonding process (e.g., heat fusion process or heat melt process). As can be appreciated, additional or alternative connection processes can be used (e.g., adhesive, mechanical connection, crimping, etc.). When the outer and inner layer portions are assembled together, the frame can be positioned between the inner and outer layer portions. When the inner and outer layer portions are secured together (e.g., reflowed, adhesive, melted seam, pressure connection, etc.) at one or both ends of the expandable sheath, the frame is also caused to be fixed in position between the inner and outer layer portions. In one non-limiting embodiment, the only one or both ends of the expandable sheath are processed so as to connect together the outer layer portion and the inner layer portion, and the mid-portion of the expandable sheath is not process to cause the outer layer portion and the inner layer portion to be connected together; however, this is not required. In such an arrangement, the frame in the mid-portion of the expandable sheath can more easily expand and contract during the expansion and contraction of the expandable sheath. In another non-limiting embodiment, the 0.1-10% (and all values and ranges therebetween) of the longitudinal length sheath is subjected to the connection process. In one non-limiting specific configuration, each end of the expandable sheath is subjected to the connection process such that each end of the expandable sheath that is process is about 0.5-2% of the longitudinal length of the expandable sheath.

In another and/or alternative non-limiting aspect of the present disclosure, the inner surface of the inner layer portion can optionally include a lubrication coating and/or liner to facilitate in the movement of a medical device through the expandable sheath. In one non-limiting embodiment, the inner surface of the inner layer portion has a coefficient of friction of no more about 0.1 (e.g., 0.0001-0.1 and all values and ranges therebetween). Non-limiting examples of lubrication materials include PTFE, polyethylene, polyvinylidene fluoride, and combinations thereof. In one non-limiting configuration, the inner surface of the inner layer portion includes a coating of PTFE.

In another and/or alternative non-limiting aspect of the present disclosure, the outer surface of the outer layer portion can optionally include a lubrication coating and/or liner to facilitate in the movement of the expandable sheath in a body passageway. In one non-limiting embodiment, the outer surface of the outer layer portion has a coefficient of friction of no more about 0.1 (e.g., 0.0001-0.1 and all values and ranges therebetween). Non-limiting examples of lubrication materials include PTFE, polyethylene, polyvinylidene fluoride, and combinations thereof. In one non-limiting configuration, the outer surface of the outer layer portion includes a coating of PTFE.

In another and/or alternative non-limiting aspect of the present disclosure, the outer surface of the outer layer portion can optionally include a hydrophilic coating to facilitate in the insertion of the expandable sheath into body passageway. Non-limiting examples of hydrophilic coatings include the Harmony™ Advanced Lubricity Coatings and other Advanced Hydrophilic Coatings available from SurModics, Inc., Eden Prairie, Minn. DSM medical coatings (available from Koninklijke DSM N.V, Heerlen, the Netherlands).

In another and/or alternative non-limiting aspect of the present disclosure, the expandable sheath optionally includes one or more radiopaque marker or fillers. The radiopaque filler or marker, when used, can be located on the outer surface of the expandable sheath, embedded on one or more layers of the expandable sheath, and/or be located between on or more layers of the expandable sheath. Non-limiting materials that can be used as a radiopaque filler or marker include barium sulfite, bismuth trioxide, titanium dioxide, and/or bismuth subcarbonate.

In another and/or alternative non-limiting aspect of the present disclosure, the inner diameter of the cavity through the longitudinal length of the expandable sheath prior to the expandable sheath being expanded is 0.08-0.2 inches (and all values and ranges therebetween), and the maximum inner diameter of the cavity through the longitudinal length of the expandable sheath after the expandable sheath being expanded is at least 0.23 inches.

In another and/or alternative non-limiting aspect of the present disclosure, the wall thickness of the expandable sheath is generally less than 0.8 mm, and typically less than 0.5 mm.

In another and/or alternative non-limiting aspect of the present disclosure, the longitudinal length of the expandable sheath is at least 5 inches and typically 5-40 inches (and all values and ranges therebetween).

In another and/or alternative non-limiting aspect of the present disclosure, the shape of the expandable sheath can optionally be tubular shaped or a cylindrical tube. The shape and size of the expandable sheath can be uniform along a majority (e.g., 60-99.99% and all values and ranges therebetween) or the full longitudinal length of the expandable sheath.

In another and/or alternative non-limiting aspect of the present disclosure, the expandable sheath in accordance with the present disclosure can be used a) to minimize trauma to a body passageway (e.g., blood vessel, etc.) by allowing for temporary expansion of a portion of the expandable sheath to accommodate a medical device and/or a delivery system for a medical device, and thereafter the expandable sheath is configured to return to its original diameter or close to its original diameter once the medical device and/or a portion of the delivery system passes through sheath, b) to reduce the length of time a procedure takes, c) to reduce the risk of a longitudinal or radial body passageway tear, d) to reduce risk of plaque dislodgement in a body passageway, e) to reduce or eliminate the need for multiple insertions sheaths or other devices for the dilation of a body passageway, f) for many types of minimally invasive surgery, such as any surgery requiring introduction of a medical device (e.g., stent, prosthetic heart valve, grafts, etc.) into a body passageway (e.g., veins, arteries, esophagus, ducts of the biliary tree, intestine, urethra, fallopian tube, other endocrine or exocrine ducts, etc.).

One non-limiting object of the disclosure, there is provided an expandable sheath.

Another and/or alternative non-limiting object of the disclosure is the provision an expandable sheath that is useable with a medical device and which expandable sheath is expandable in at least the regions that the medical device is positioned in the expandable sheath and which sheath reforms to its same or similar size and shape after the medical device has passed through a portion of all of the expandable sheath.

Another and/or alternative non-limiting object of the disclosure is the provision an expandable sheath that can be used to protect the body passageway of a patient as a medical device is inserted into and/or through the body passageway and to a treatment site.

Another and/or alternative non-limiting object of the disclosure is the provision an expandable sheath that can be used with catheter arrangements for repairing and/or replacing heart valves.

Another and/or alternative non-limiting object of the disclosure is the provision an expandable sheath that can be used to reduce or minimize trauma to a body passageway (e.g., blood vessel, duct, etc.) the vessel by allowing for temporary expansion of a portion of the expandable sheath to accommodate at least a portion of the delivery system and/or medical device, followed by a return to the same or similar original diameter once the delivery system and/or medical device has partially or fully passes through the expandable sheath.

Another and/or alternative non-limiting object of the disclosure is the provision an expandable sheath that can be configured to progressively increase in outer diameter and then decrease in outer diameter as the medical device moves through the interior passageway of the expandable sheath.

Another and/or alternative non-limiting object of the disclosure is the provision an expandable sheath that can be configured such that the outer diameter or cross-sectional area of the expandable sheath and the diameter or cross-sectional area of the interior passageway expands to a second diameter along the longitudinal length of the expandable sheath as the medical device moves through the expandable sheath, and then partially or fully returns to the first outer diameter once the medical device has fully passes through the expandable sheath.

Another and/or alternative non-limiting object of the disclosure is the provision an expandable sheath that is partially for fully formed of a shape memory material that is used to facilitate in causing the expandable sheath to partially or fully return to the first outer diameter once the medical device has partially or fully passed through the expandable sheath.

Another and/or alternative non-limiting object of the disclosure is the provision an expandable sheath that provides access to a body passageway for delivering a medical device into the body passageway; the expandable sheath expandable from a first diameter to a second diameter; the second diameter greater than the first diameter; the expandable sheath including an internal cavity that extends a longitudinal length of the expandable sheath; the expandable sheath includes a plurality of thin bands and plurality of thick bands that extend along a longitudinal length of the expandable sheath; each of the thin bands include first and second ends; each of the thick bands includes first and second ends; the first end of the thick bands and thin bands are connected together; the second end of the thick bands and thin bands are connected together; at least one of the thin bands configured to increase in longitudinal length when the expandable sheath expands from the first diameter to the second diameter.

Another and/or alternative non-limiting object of the disclosure is the provision an expandable sheath that is configured to expand to the second diameter during movement of the medical device through the internal cavity of the expandable sheath; the expandable device configured to contract from the second diameter to the first diameter after the medial device partially or fully passes through the internal cavity of the expandable sheath.

Another and/or alternative non-limiting object of the disclosure is the provision an expandable sheath wherein one or more of the thin bands is partially or fully formed of a shape memory material.

Another and/or alternative non-limiting object of the disclosure is the provision an expandable sheath wherein one or more of the thin bands is formed of a different material from one or more of the thick bands.

Another and/or alternative non-limiting object of the disclosure is the provision an expandable sheath wherein a) one or more of the thin bands has an S-shaped configuration when the expandable sheath in the first diameter, and wherein one or more of the thin bands is reshaped from the S-shape configuration to an arc-shape when the expandable sheath expands from the firsts diameter to the second diameter, or b) one or more of the thin bands have overlapping portions when the expandable sheath in the first diameter, and wherein one or more of the thin bands is reshaped from being in an overlapping configuration to a non-overlapping configuration when the expandable sheath expands from the firsts diameter to the second diameter.

Another and/or alternative non-limiting object of the disclosure is the provision an expandable sheath including a tapered transition having a variable thickness located at end portion of one or more of the thin bands and terminates at the first or second end of the thick band.

Another and/or alternative non-limiting object of the disclosure is the provision an expandable sheath wherein a circumferential length of the one or more of the thin bands is equal to or greater than a circumferential length of the one or more of the thick bands when the expandable sheath is in the second diameter.

Another and/or alternative non-limiting object of the disclosure is the provision an expandable sheath wherein the thin bands and/or the thick bands is partially or fully formed of a polymer material.

Another and/or alternative non-limiting object of the disclosure is the provision an expandable sheath wherein the polymer material includes one or more polymers selected from the group consisting of polytetrafluoroethylene (PTFE), polyimide, polyetheretherketone (PEEK), polyurethane, nylon, polyethylene, polyamide, styrenic block copolymer (SBC) (e.g., polystyrene blocks and rubber blocks [e.g., polybutadiene, polyisoprene or their hydrogenated equivalents]), polyether block amides, polyether block ester copolymer, thermoset silicone, latex, poly-isoprene rubbers, high density polyethylene (HDPE), polyurethane resin, elastomers formed of block copolymers made up of rigid polyamide blocks and soft polyether blocks.

Another and/or alternative non-limiting object of the disclosure is the provision an expandable sheath wherein the thin bands and/or the thick bands is partially or fully formed of a metal material.

Another and/or alternative non-limiting object of the disclosure is the provision an expandable sheath wherein the metal material includes one or more material selected from the group consisting of a) copper-aluminum-nickel alloy, b) nickel-titanium alloy, c) zinc-copper-gold-iron alloy, d) Fe—Mn—Si alloy, e) Cu—Zn—Al alloy, f) Cu—Al—Ni alloy, g) Ag—Cd alloy, h) Au—Cd alloy, i) Co—Ni—Al alloy, j) Co—Ni—Ga alloy, k) Cu—Al—Be—X (X═Zr, B, Cr, Gd) alloy, 1) Cu—Al—Ni—Hf alloy, m) Cu—Sn alloy, n) Cu—Zn alloy, o) Cu—Zn—X (X═Si, Al, Sn) alloy, p) Fe—Pt alloy, q) Mn—Cu alloy, r) Ni—Fe—Ga alloy, s) Ni—Ti—Hf alloy, t) Ni—Ti—Pd alloy, u) Ni—Mn—Ga alloy, v) Ni—Mn—Ga—Cu alloy, w) Ni—Mn—Ga—Co alloy, and x) Ti—Nb alloy.

Another and/or alternative non-limiting object of the disclosure is the provision an expandable sheath wherein a plurality or all of the thick bands have the same shape, size, configuration and/or composition.

Another and/or alternative non-limiting object of the disclosure is the provision an expandable sheath wherein a plurality or all of the thin bands have the same shape, size, configuration and/or composition.

Another and/or alternative non-limiting object of the disclosure is the provision an expandable sheath wherein a total volume of the thick bands is about 30-80 vol. % of the expandable sheath.

Another and/or alternative non-limiting object of the disclosure is the provision an expandable sheath wherein a thinnest thickness of one or more the thin bands is 10-80% of a thickness of a maximum thickness of one or more of the thick bands.

Another and/or alternative non-limiting object of the disclosure is the provision an expandable sheath wherein 10-49 wt. % of the expandable sheath is formed of one type of polymer and 51-90 wt. % of the expandable sheath is formed of another polymer.

Another and/or alternative non-limiting object of the disclosure is the provision an expandable sheath wherein the expandable sheath in the fully contracted orientation has a cross-sectional wall thickness that is generally the same for 70%-98% of a circumference of the expandable sheath.

Another and/or alternative non-limiting object of the disclosure is the provision an expandable sheath wherein the expandable sheath includes one or more radiopaque fillers or markers.

Another and/or alternative non-limiting object of the disclosure is the provision an expandable sheath wherein the expandable sheath is formed by an extrusion process, stamping process, or casting process.

Another and/or alternative non-limiting object of the disclosure is the provision a method of introducing a prosthetic heart valve into a patient's vasculature, the method comprising: a) inserting an expandable sheath at least partially into a blood vessel of a patient; the expandable sheath expandable from a first diameter to a second diameter; the second diameter greater than the first diameter; the expandable sheath including an internal cavity that extends a longitudinal length of the expandable sheath; the expandable sheath includes a plurality of thin bands and plurality of thick bands that extend along a longitudinal length of the expandable sheath; each of the thin bands include first and second ends; each of the thick bands includes first and second ends; the first end of the thick bands and thin bands are connected together; the second end of the thick bands and thin bands are connected together; at least one of the thin bands configured to increase in longitudinal length when the expandable sheath expands from the first diameter to the second diameter; and b) advancing a prosthetic heart valve through the internal cavity of the expandable sheath so as to cause the expandable sheath to expand from the first diameter to the second diameter as the prosthetic heart valve partially or fully passes through the internal cavity of the expandable sheath.

Another and/or alternative non-limiting object of the disclosure is the provision method of introducing a prosthetic heart valve into a patient's vasculature wherein the prosthetic heart valve is a stent mounted heart valve mounted in a radially crimped state on a delivery apparatus.

Another and/or alternative non-limiting object of the disclosure is the provision method of introducing a prosthetic heart valve into a patient's vasculature wherein the expandable sheath contracts from the second diameter to the first diameter after the prosthetic heart valve has partially or fully passed through the internal cavity of the expandable sheath.

Another and/or alternative non-limiting object of the disclosure is the provision that a plurality or all of the thick bands have the same shape, size, configuration and/or composition.

Another and/or alternative non-limiting object of the disclosure is the provision that a plurality or all of the thin bands have the same shape, size, configuration and/or composition.

Another and/or alternative non-limiting object of the disclosure is the provision that a total volume of the thin bands constitutes 10-60% of a plurality or all of the thick bands have the same shape, size, configuration, and/or composition.

Another and/or alternative non-limiting object of the disclosure is the provision that a total outer surface of the thick bands constitutes about 30-80% of an outer circumference of the expandable sheath when the expandable sheath is in the fully expanded orientation.

Another and/or alternative non-limiting object of the disclosure is the provision that the polymer material constitutes 60-100% of the expandable sheath.

Another and/or alternative non-limiting object of the disclosure is the provision that the expandable sheath in the fully expanded orientation has a cross-sectional wall thickness that is generally the same for 5-65% of the circumference of the expandable sheath.

Another and/or alternative non-limiting object of the disclosure is the provision that the expandable sheath has a constant cross-sectional size and/or shape along 50-100% of a longitudinal length of the expandable sheath when the expandable sheath is in the fully contracted orientation and/or when the expandable sheath is in the fully expanded orientation.

Another and/or alternative non-limiting object of the disclosure is the provision that the expandable sheath includes a plurality of segments that have overlapping portions that are each configured to locally, partially or fully move to a non-overlapping orientation, thereby increasing the length of such segments when the expandable sheath is expanded from the fully contracted orientation to the fully expanded orientation.

Another and/or alternative non-limiting object of the disclosure is the provision that an outer cross-sectional profile of the expandable sheath in the fully expanded orientation and/or the fully contracted orientation is generally circular.

Another and/or alternative non-limiting object of the disclosure is the provision that the cross-sectional shape of an internal cavity of the expandable sheath in the fully contracted orientation is generally circular.

Another and/or alternative non-limiting object of the disclosure is the provision that the outer layer is coextruded over one or more portions of one or more of the bands.

Another and/or alternative non-limiting object of the disclosure is the provision of a method of introducing a prosthetic heart valve into a patient's vasculature, the method comprising: a) inserting an expandable sheath at least partially into a blood vessel of a patient; and b) advancing a prosthetic heart valve through a central lumen of a delivery sheath so as to cause the delivery sheath to expand from an outwardly directed radial force of the prosthetic heart valve exerted against the expandable sheath, and then at least partially contract as the prosthetic heart valve passes through the expandable sheath.

Another and/or alternative non-limiting object of the disclosure is the provision that the prosthetic heart valve is a stent mounted heart valve mounted in a radially crimped state on a delivery apparatus.

Another and/or alternative non-limiting object of the disclosure is the provision an expandable sheath that provides access to the vasculature for delivering of medical devices or for medical intervention, the expandable sheath includes: a) a plurality of bands, each of the bands including first and second ends; and b) an outer layer that partially or fully encases one or more of the longitudinal or radial bands; and wherein the plurality of bands and/or the outer layer are formed of a polymer or metallic material; and wherein the first end of each of the bands is bonded to, connected to, encased or merges with an adjacently positioned band; and wherein the second end of each of the bands is bonded to, connected to, encased or merges with an adjacently positioned band; and wherein the outer layer is bonded to, connected to, encases or merges one or more of the bands; wherein the expandable sheath is expandable between a fully contracted orientation and a fully expanded orientation; and wherein the expandable sheath opens for the delivery or removal of medical devices or for medical intervention and closes and/or retracts to a smaller diameter once medical devices distally pass or is proximally removed from the expandable sheath or for medical intervention.

Another and/or alternative non-limiting object of the disclosure is the provision an expandable sheath that is adapted for use in the delivery of a medical device into a body passageway; the sheath comprising a) an outer layer portion; b) an inner layer portion; and c) a frame; the frame at least partially positioned between the inner and outer layer portions; and wherein the sheath is configured to expand from a first outer diameter to a second outer diameter as the medical device is moved through the sheath, and thereafter the sheath partially or fully returns to the first outer diameter once the medical device has passed partially or fully through the sheath.

Another and/or alternative non-limiting object of the disclosure is the provision an expandable sheath that includes an internal cavity extending the longitudinal length of the sheath.

Another and/or alternative non-limiting object of the disclosure is the provision an expandable sheath that has a cylindrical shape.

Another and/or alternative non-limiting object of the disclosure is the provision an expandable sheath wherein the outer layer portion includes a polymer material.

Another and/or alternative non-limiting object of the disclosure is the provision an expandable sheath wherein the outer layer portion includes a first and second polymer layer.

Another and/or alternative non-limiting object of the disclosure is the provision an expandable sheath wherein the first and second polymer layers of the outer layer are formed of a different polymer

Another and/or alternative non-limiting object of the disclosure is the provision an expandable sheath wherein the first and second polymer layers of the outer layer are co-extruded.

Another and/or alternative non-limiting object of the disclosure is the provision an expandable sheath wherein a thickness of the outer layer portion is greater than a thickness of the inner layer portion.

Another and/or alternative non-limiting object of the disclosure is the provision an expandable sheath that wherein an outer surface of the outer layer portion includes a friction reducing coating or layer; the friction reducing coating or layer optionally has a coefficient of friction of no more than 0.1.

Another and/or alternative non-limiting object of the disclosure is the provision an expandable sheath wherein the inner layer portion includes a polymer material.

Another and/or alternative non-limiting object of the disclosure is the provision an expandable sheath wherein the inner layer portion includes a first and second polymer layer.

Another and/or alternative non-limiting object of the disclosure is the provision an expandable sheath wherein the first and second polymer layers of the inner layer are formed of a different polymer.

Another and/or alternative non-limiting object of the disclosure is the provision an expandable sheath wherein the first and second polymer layers of the inner layer are co-extruded.

Another and/or alternative non-limiting object of the disclosure is the provision an expandable sheath wherein an inner surface of the inner layer portion includes a friction reducing coating or layer; the friction reducing coating or layer has a coefficient of friction of optionally no more than 0.1.

Another and/or alternative non-limiting object of the disclosure is the provision an expandable sheath wherein the frame includes a shape memory material.

Another and/or alternative non-limiting object of the disclosure is the provision an expandable sheath that wherein the shape memory material includes a nickel-titanium alloy.

Another and/or alternative non-limiting object of the disclosure is the provision an expandable sheath that wherein the frame is at least partially positioned between the inner and outer layer portions.

Another and/or alternative non-limiting object of the disclosure is the provision an expandable sheath that wherein the frame includes first and second frame wires that extend along a majority of the longitudinal length of the sheath; a majority of a longitudinal length of each of the first and second frame wires do not overlap one another along a majority of the longitudinal length of the sheath.

Another and/or alternative non-limiting object of the disclosure is the provision an expandable sheath wherein the first and second frame wires include a plurality of undulations along a majority of the longitudinal length of each of the first and second frame wires.

Another and/or alternative non-limiting object of the disclosure is the provision an expandable sheath including longitudinal shaping structure; the longitudinal shaping structure configured to inhibit kinking of the sheath.

Another and/or alternative non-limiting object of the disclosure is the provision an expandable sheath wherein the longitudinal shaping structure is at least partially located in the outer layer portion.

Another and/or alternative non-limiting object of the disclosure is the provision an expandable sheath wherein the longitudinal shaping structure is positioned along a majority of the longitudinal length of the sheath.

Another and/or alternative non-limiting object of the disclosure is the provision an expandable sheath wherein the longitudinal shaping structure include shape memory material.

Another and/or alternative non-limiting object of the disclosure is the provision an expandable sheath wherein the shape memory material of the longitudinal shaping structure includes nickel-titanium alloy.

Another and/or alternative non-limiting object of the disclosure is the provision an expandable sheath wherein the longitudinal shaping structure as a thickness of 0.002-0.03 inches.

Another and/or alternative non-limiting object of the disclosure is the provision an expandable sheath wherein the longitudinal shaping structure includes a first shaping structure; the first shaping structure having a straight profile along a majority of a longitudinal length of the first shaping structure; the first shaping structure extending along a majority of the longitudinal length of the sheath.

Another and/or alternative non-limiting object of the disclosure is the provision an expandable sheath wherein the first shaping structure has a rod shape configuration.

Another and/or alternative non-limiting object of the disclosure is the provision an expandable sheath wherein the longitudinal shaping structure includes a second shaping structure; the second shaping structure having a straight profile along a majority of a longitudinal length of the second shaping structure; the second shaping structure extending along a majority of the longitudinal length of the sheath; the second shaping structure spaced from the first shaping structure.

Another and/or alternative non-limiting object of the disclosure is the provision an expandable sheath that wherein a first end of the sheath is subjected to a reflow process to connect together the inner and outer layer portions; a majority of the longitudinal length of the sheath not subjected to the reflow process.

Another and/or alternative non-limiting object of the disclosure is the provision an expandable sheath that wherein a second end of the sheath is subjected to a reflow process to connect together the inner and outer layer portions; a majority of the longitudinal length of the sheath not subjected to the reflow process.

Another and/or alternative non-limiting object of the disclosure is the provision of a method of introducing a medical device into a body passageway of a patient; the method comprising: a) providing an expandable sheath; b) inserting the sheath at least partially into the body passageway; and c) advancing the medical device through the sheath to cause the sheath to locally expand from a non-expanded configuration to an expanded configuration due to movement of the medical device through the sheath, and thereafter the sheath contracts back to the non-expanded configuration as the medical device through the sheath.

Another and/or alternative non-limiting object of the disclosure is the provision of a method of introducing a medical device into a body passageway of a patient wherein the step of inserting includes passing the sheath transcutaneously through a surgically-created opening in the patient's skin such that at least a portion of the outer payer portion of the sheath is positioned adjacent to the surgically-created opening.

Another and/or alternative non-limiting object of the disclosure is the provision of a method of introducing a medical device into a body passageway of a patient further including the step of implanting the medical device at a treatment site within the patient.

Another and/or alternative non-limiting object of the disclosure is the provision of a method of introducing a medical device into a body passageway of a patient wherein the medical device in a stent mounted heart valve mounted in a radially crimped state on a delivery apparatus.

Another and/or alternative non-limiting object of the disclosure is the provision of a method of introducing a medical device into a body passageway of a patient further including the step of expanding the medical device at the treatment site of the patient.

Other aspects, advantages and novel features of the present disclosure will become apparent from the following detailed description and figures of the disclosure.

### BRIEF DESCRIPTION OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. Reference may now be made to the drawings, which illustrate various embodiments that the disclosure may take in physical form and in certain parts and arrangement of parts wherein:

FIG. 1 illustrates an expandable sheath in accordance with the present disclosure that can be used with a delivery apparatus for facilitating in delivering a medical device into a patient.

FIGS. 2-5 illustrate one non-limiting configuration of an expandable sheath in accordance with the present disclosure.

FIG. 6 illustrates a modification to the expandable sheath illustrated in FIGS. 2-5.

FIG. 7 illustrates another modification to the expandable sheath illustrated in FIGS. 2-6.

FIGS. 8A-8B illustrated another modification to the expandable sheath illustrated in FIGS. 2-7.

FIGS. 9A-9C illustrate a front-end view of another non-limiting configuration of an expandable sheath in accordance with the present disclosure.

FIGS. 10A-10C illustrate a front-end view of another non-limiting configuration of an expandable sheath in accordance with the present disclosure.

### DESCRIPTION OF NON-LIMITING EMBODIMENTS OF THE DISCLOSURE

A more complete understanding of the articles/devices, processes and components disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

For the sake of simplicity, the attached figures may not show the various ways (readily discernable, based on this disclosure, by one of ordinary skill in the art) in which the disclosed system, method and apparatus can be used in combination with other systems, methods and apparatuses. Additionally, the description sometimes uses terms such as “produce” and “provide” to describe the disclosed method. These terms are abstractions of the actual operations that can be performed. The actual operations that correspond to these terms can vary depending on the particular implementation and are, based on this disclosure, readily discernible by one of ordinary skill in the art.

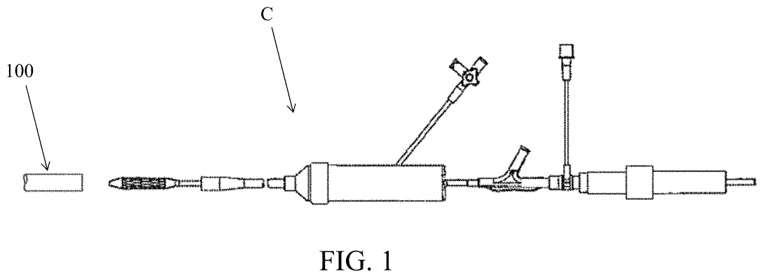

Referring now to FIG. 1, there is illustrate an expandable sheath 100 in accordance with the present disclosure used use with a representative delivery apparatus C for delivering a medical device such as a TARV to the heart of a patient. The delivery apparatus can optionally include a steerable guide catheter (also referred to as a flex catheter), a balloon catheter extending through the guide catheter, and an optional nose catheter extending through the balloon catheter. The guide catheter, the balloon catheter, and the nose catheter in the illustrated non-limiting embodiment can be configured to slide longitudinally relative to each other to facilitate delivery and positioning of the medical device at a treatment site in the patient. The expandable sheath 100 can be inserted into a blood vessel such that one end of the expandable sheath is inserted into the blood vessel. The expandable sheath 100 can optionally include a valve (e.g., hemostasis valve) at the opposite end of the expandable sheath. The delivery apparatus C can be inserted into the expandable sheath 100, and the medical device can then be delivered and implanted within patient at the treatment site in the patient.

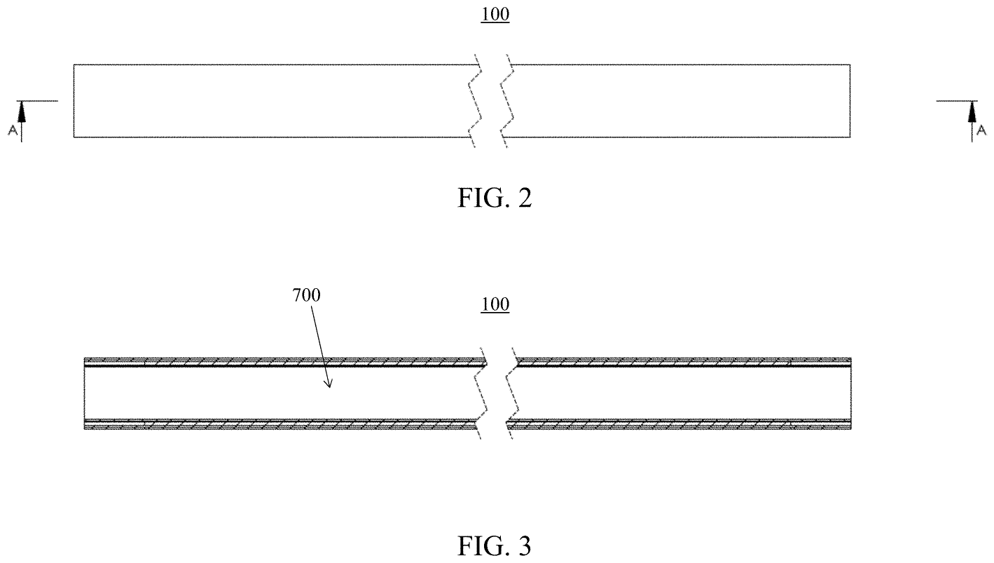

Referring now to FIGS. 2-8 several non-limiting embodiments of the expandable sheath 100 in accordance with the present disclosure are illustrated. As illustrated in FIGS. 3, 4 and 7, the expandable sheath 100 includes multiple layers. The expandable sheath 100 includes an outer layer portion 300, a frame 400 and an inner layer portion 200. The outer layer portion 300, a frame 400 and an inner layer portion 200 facilitate in maintaining the shape and integrity of the expandable sheath 100.

The expandable sheath 100 includes an inner layer portion 200 that can be formed of one or more polymer layers 210, 220. The one or more polymer layers used to partially or fully form the inner layer portion can include polyethylene, polytetrafluoroethylene, polyimide, polyetheretherketone, polyurethane, nylon, polyether block amides, polyether block ester copolymer, thermoset silicone, latex, poly-isoprene rubbers, styrene ethylene butylene styrene, polyesters, fluoropolymers, polyvinyl chloride, polyolefin, and/or high-density polyethylene. In one non-limiting configuration, the inner layer portion is or includes polyethylene. The inner layer portion is formed of a single polymer layer, or can be formed of at least two polymer layers. When the inner layer portion includes two or more layers, the composition of the layers can be the same or different. In one non-limiting configuration, the inner layer portion is formed of two layers, and wherein the inner layer 210 is formed of high-density polyethylene and the outer layer 220 is formed of styrene ethylene butylene styrene, and wherein the two layers are optionally co-extruded, and wherein the inner layer forms 30-70% of the total thickness of the inner layer portion and the outer layer forms 30-70% of the total thickness of the inner layer portion. In another non-limiting embodiment, the thickness of the inner layer portion is generally at least 0.002 inches, typically 0.002-0.02 inches, and more typically 0.004-0.008 inches. In another non-limiting embodiment, the inner layer portion is formed of a material that allows the outer diameter or outer cross-sectional area of the expandable sheath 100 and the diameter or cross-sectional area of the interior passageway to expand and contract as the medical device is moved through the expandable sheath 100 without damaging the material that forms inner layer portion. In another non-limiting embodiment, when the inner layer portion is formed of two or more layers, the inner layer portion can optionally be formed by a co-extrusion process.

The expandable sheath includes an outer layer portion 300 that can be formed of one or more polymer layers 310, 320. The outer layer portion can be formed of the same or similar material as the inner layer portion. In one non-limiting embodiment, the one or more polymer layers used to partially or fully form the outer layer portion can include polyethylene, polytetrafluoroethylene, polyimide, polyetheretherketone, polyurethane, nylon, polyether block amides, polyether block ester copolymer, thermoset silicone, latex, poly-isoprene rubbers, styrene ethylene butylene styrene, polyesters, fluoropolymers, polyvinyl chloride, polyolefin, and/or high-density polyethylene. In one non-limiting configuration, the outer layer portion is or includes polyethylene. The outer layer portion is formed of a single polymer layer, or formed of at least two polymer layers. When the outer layer portion includes two or more layers, the composition of the layers can be the same or different. In one non-limiting configuration, the outer layer portion is formed of two layers, and wherein the outer layer 310 is formed of high-density polyethylene and the inner layer 320 is formed of styrene ethylene butylene styrene, and wherein the two layers are optionally co-extruded, and wherein the inner layer forms 30-70% of the total thickness of the outer layer portion and the outer layer forms 30-70% of the total thickness of the outer layer portion. In another non-limiting embodiment, the thickness of the outer layer portion is generally at least 0.002 inches, typically 0.002-0.03 inches, and more typically 0.004-0.015 inches. In another non-limiting embodiment, the thickness of the outer layer portion is greater than the thickness of the inner layer portion. In another non-limiting embodiment, the outer layer portion is formed of a material that allows the outer diameter or outer cross-sectional area of the expandable sheath and the diameter or cross-sectional area of the interior passageway to expand and contract as the medical device is moved through the expandable sheath without damaging the material that forms outer layer portion. In another non-limiting embodiment, when the outer layer portion is formed of two or more layers, the outer layer portion can optionally be formed by a co-extrusion process.

The expandable sheath 100 includes a frame 400 that includes a shape memory material. In one non-limiting embodiment, the majority (e.g., 60-99.99% and all values and ranges therebetween) or all of the frame is formed of a shape member material. The shape member material is or includes a) copper-aluminum-nickel alloy, b) nickel-titanium alloy, c) zinc-copper-gold-iron alloy, d) Fe—Mn—Si alloy, e) Cu—Zn—Al alloy, f) Cu—Al—Ni alloy, g) Ag—Cd alloy, h) Au—Cd alloy, i) Co—Ni—Al alloy, j) Co—Ni—Ga alloy, k) Cu—Al—Be—X (X═Zr, B, Cr, Gd) alloy, l) Cu—Al—Ni—Hf alloy, m) Cu—Sn alloy, n) Cu—Zn alloy, o) Cu—Zn—X (X═Si, Al, Sn) alloy, p) Fe—Pt alloy, q) Mn—Cu alloy, r) Ni—Fe—Ga alloy, s) Ni—Ti—Hf alloy, t) Ni—Ti—Pd alloy, u) Ni—Mn—Ga alloy, v) Ni—Mn—Ga—Cu alloy, w) Ni—Mn—Ga—Co alloy, x) Ti—Nb alloy, y) polyacrylate-based SMPs (e.g., t-butylacrylate-co-poly(ethyleneglycol) dimethacrylate (tBA-co-PEGDMA) polymers, etc.), z) (meth)acrylate-based SMPs, aa) polyurethane-based SMPs, and/or bb) blends of polyurethane and polyvinylchloride-based SMPs. In one non-limiting configuration, the frame is partially or fully formed of nickel-titanium alloy.

The thickness of the frame 400 of the expandable sheath 100 is generally at least 0.002 inches, typically 0.002-0.03 inches, and more typically 0.004-0.012 inches. In another non-limiting embodiment, the thickness of the frame is greater than the thickness of the inner layer portion. In another non-limiting embodiment, the thickness of the frame is equal to or less than the thickness of the outer layer portion.

The frame 400 of the expandable sheath is formed of a material that allows the outer diameter or outer cross-sectional area of the expandable sheath 100 and the diameter or cross-sectional area of the interior passageway to expand and contract as the medical device is moved through the expandable sheath 100 without damaging the frame.

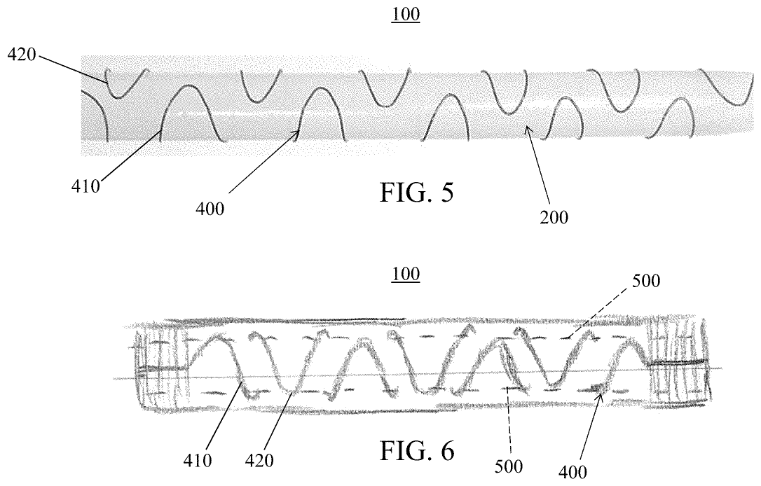

The frame 400 of the expandable sheath 100 has a configuration that allows the outer diameter or outer cross-sectional area of the expandable sheath 100 and the diameter or cross-sectional area of the interior passageway to expand and contract as the medical device is moved through the expandable sheath 100 without damaging the frame. In one non-limiting embodiment, the frame provides strength, structure and/or shape to the expandable sheath 100. In another non-limiting embodiment, the frame partially or fully includes a non-overlapping structure (e.g., few or no interlocking fingers or struts, etc.). In another non-limiting embodiment, the frame includes two sets of undulating wires along the longitudinal length of the expandable sheath. One non-limiting configuration of undulating wires in the frame are illustrated in FIGS. 5, 6 and 8A. As illustrated in FIGS. 5, 6 and 8A, frame 400 includes undulating wires 410 and 420 that are configured to not overlap one another along the partial or full longitudinal length of the expandable sheath 100. As can be appreciated, the frame can include a single wire or more than two non-wires, wherein the one or more wires of the frame 400 do not overlap along 60-100% of the longitudinal length of the expandable sheath 100.

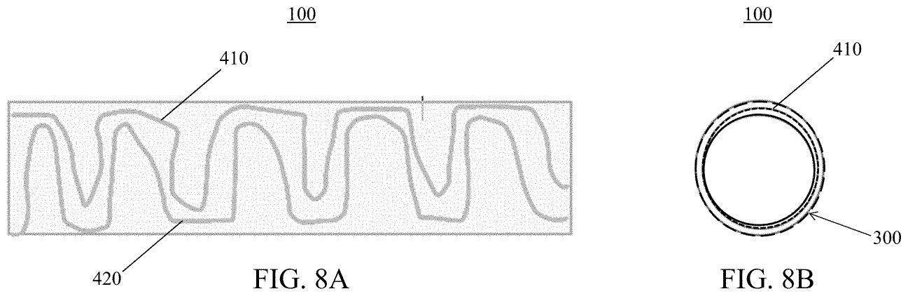

The one or more wires can be uniformly spaced from the inner surface of the inter layer portion 200, or vary in distance from the inner surface of the inter layer portion 200 as illustrated in FIG. 8B. FIG. 8B is a view of the front face or front end of an expandable sheath 100.

Referring again to FIGS. 5 and 6, the two wires 410, 420 of the frame 400 each have a generally sinusoidal shape. Generally, the two wire have the same shape; however, this is not required. As illustrated in FIG. 8A, the two wires 410, 420 of the frame 400 have a non-uniform wavy shape along the longitudinal length of the expandable sheath 100. The pattern and shape of the one or more wires of the frame 400 enables the outer diameter or outer cross-sectional area of the expandable sheath 100 and the diameter or cross-sectional area of the interior passageway of the expandable sheath 100 to expand and contract as a medical device is moved through the expandable sheath 100 without damaging the frame 400 of the expandable sheath 100. The shape memory material used to at least partially form the frame 400 causes the expandable sheath 100 to contact to its original or near original shape after being expanded as the medical device passes through the expandable sheath 100. Generally, the material used to form each of the wires of the frame 400 is the same; however, this is not required. Generally, the shape and size of each of the wires of the frame 400 are the same; however, this is not required.

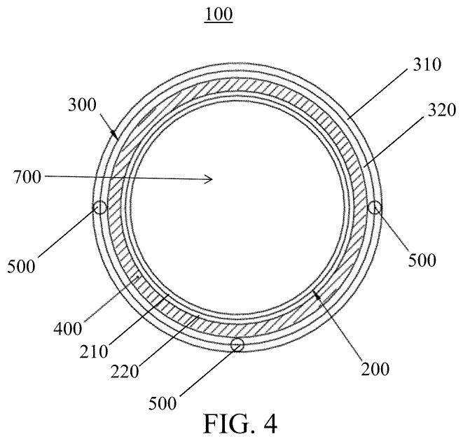

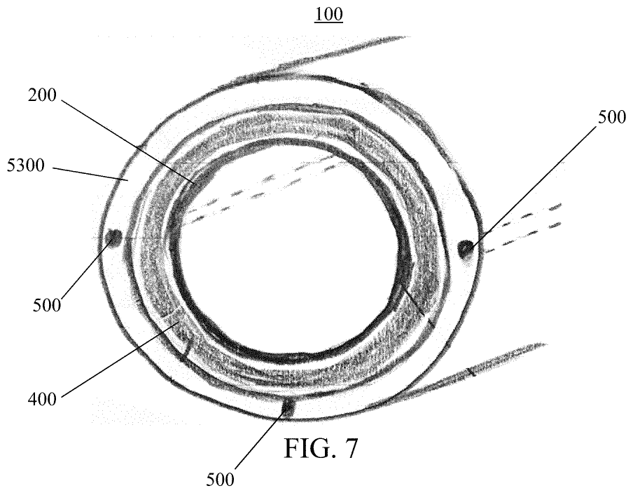

The expandable sheath can optionally include one or more longitudinal shaping structures 500 positioned along the longitudinal length of the expandable sheath 100. The one or more longitudinal shaping structures 500 are configured to inhibit or prevent kinking of the expandable sheath 100 during use and during insertion and removal of the expandable sheath 100 from a body passageway. The one or more longitudinal shaping structures 500 can also or alternatively be used to inhibit or prevent the compressing and/or elongation of the expandable sheath 100 while it a) expands in diameter, b) reduces in diameter, c) is inserted into a body passageway, or d) is removed from the body passageway. As illustrated in FIGS. 4, 6 and 7, the longitudinal shaping structures 500 are in the form of one or more wires that extend partially or fully along the longitudinal length of the expandable sheath 100. When two of more wires are used, the wires are generally shape from one another. As illustrated in FIGS. 4, 6 and 7, the longitudinal shaping structures 500 are the form of three spaced wires that extend along 90-100% the longitudinal length of the expandable sheath 100. The spacing of the three wires from one another are about a 20-30% of the outer circumference of the expandable sheath 100. As illustrated in FIGS. 4 and 7, there is a larger gap or spacing between two of the wires. This gap is about 40-60% of the outer circumference of the expandable sheath 100. This larger gaping or spacing is optional. When such gap or spacing exists, it facilitates in the bending of the expandable sheath 100 in the location absent the wire. As can be appreciated, the use of a single wire or two equally shaped wires could also be used for easier bending of the expandable sheath 100. One or more longitudinal shaping structures can be partially or fully formed of a shape memory material. The shape memory material used to partially or fully form the one or more longitudinal shaping structures 500 can be used to cause the expandable sheath 100 to straighten along its longitudinal axis, thereby inhibiting or preventing kinking of the expandable sheath 100 when inserted and/or removed from a body passageway. The shape memory material can be the same or similar material as described above with regard to the frame 400. In one non-limiting configuration, the one or more longitudinal shaping structures 500 are partially or fully formed of a nickel-titanium alloy. As illustrated in FIGS. 4 and 7, the one or more longitudinal shaping structures 500 are positioned in the outer layer portion 300 of the expandable sheath 100. In one non-limiting configuration, the one or more longitudinal shaping structures 500 are encapsulated in the outer layer portion of the expandable sheath. The one or more longitudinal shaping structures can be encapsulated between the inner and outer layers 310, 320 of the outer layer portion 300 of the expandable sheath 100; however, this is not required. The one or more longitudinal shaping structures 500 can be coextruded with the outer layer portion 300; however, other processes can be used to secure the one or more longitudinal shaping structures 500 to and/or in the outer layer portion 300 (e.g., adhesive, mechanical connection, etc.). After the one or more longitudinal shaping structures 500 are positioned between layers of the outer layer portion 300, the outer layer position 300 can be optionally reflowed and/or the layers are optionally adhesively connected together to secure together the layers of the outer layer portion 300. Such securing together of the layers facilitates in maintaining the one or more longitudinal shaping structures 500 in position in the expandable sheath 100. When the one or more longitudinal shaping structures 500 are in the form of a wire, the size and shape of the wire can be the same or similar to the size and shape of the wire used to form the frame 400; however, this is not required. The one or more longitudinal shaping structures 500 are generally spaced from the frame 400 and/or spaced from the inner layer portion 200. Although not shown, one or more or all of the longitudinal shaping structures 500 can optionally be located in the inner layer portion 200.

One or both ends of the expandable sheath 100 can optionally be subjected to a connection processed so as to connect together the outer layer portion 300 and the inner layer portion 200. One non-limiting connection process can optionally be a reflow process to secure the outer layer portion 300 and the inner layer portion 200 by a heat bonding process (e.g., heat fusion process or heat melt process). As can be appreciated, additional or alternative connection processes can be used (e.g., adhesive, mechanical connection, crimping, etc.). As illustrated in FIGS. 4 and 7, when the outer and inner layer portions 300, 200 are assembled together, the frame 400 can be optionally positioned between the inner and outer layer portions 200, 300. As can be appreciated, the frame 400 can be partially or fully positioned in the inner layer portion 200 and/or the outer layer portion 300. When the inner and outer layer portions 200, 300 are secured together at one or both ends of the expandable sheath 100, the frame 400 is also caused to be fixed in position between the inner and outer layer portions 200, 300. Generally, only one or both ends of the expandable sheath 100 are processed so as to connect together the outer layer portion 300 and the inner layer portion 200, and the mid-portion of the expandable sheath 100 is not process to cause the outer layer portion 300 and the inner layer portion 200 to be connected together; however, this is not required. In such an arrangement, the frame 400 in the mid-portion of the expandable sheath 100 can more easily expand and contract during the expansion and contraction of the expandable sheath 100 since the frame 400 is not secured in the mid-portion of the expandable sheath 100 to the inner layer portion 200 and/or the outer layer portion 300, thus allowing the frame to move relative to the inner layer portion 200 and/or the outer layer portion 300 when the expandable sheath 100 expands and/or contracts in diameter. Generally, 0.1-10% of the longitudinal length expandable sheath 100 is subjected to the connection process. In one non-limiting specific configuration, each end of the expandable sheath 100 is subjected to the connection process such that each end of the expandable sheath 100 that is process is about 0.5-2% of the longitudinal length of the expandable sheath 100.

The inner surface of the inner layer portion 200 can optionally include a lubrication coating and/or liner to facilitate in the movement of a medical device through the expandable sheath 100. In one non-limiting embodiment, the inner surface of the inner layer portion has a coefficient of friction of no more about 0.1 (e.g., 0.0001-0.1 and all values and ranges therebetween). Non-limiting examples of lubrication materials include PTFE, polyethylene, polyvinylidene fluoride, and combinations thereof. In one non-limiting configuration, the inner surface of the inner layer portion includes a coating of PTFE.

The outer surface of the outer layer portion 300 can optionally include a lubrication coating and/or liner to facilitate in the movement of the expandable sheath 100 into and/or out of a body passageway. In one non-limiting embodiment, the outer surface of the outer layer portion 300 has a coefficient of friction of no more about 0.1 (e.g., 0.0001-0.1 and all values and ranges therebetween). Non-limiting examples of lubrication materials include PTFE, polyethylene, polyvinylidene fluoride, and combinations thereof. In one non-limiting configuration, the outer surface of the outer layer portion includes a coating of PTFE.

The outer surface of the outer layer portion 300 can optionally include a hydrophilic coating to facilitate in the insertion of the expandable sheath 100 into and/or out of a body passageway. Non-limiting examples of hydrophilic coatings include the HarmonyTM Advanced Lubricity Coatings and other Advanced Hydrophilic Coatings available from SurModics, Inc., Eden Prairie, Minn. DSM medical coatings (available from Koninklijke DSM N.V, Heerlen, the Netherlands).

The inner diameter of the cavity 700 through the longitudinal length of the expandable sheath 100 prior to the expandable sheath 100 being expanded can be 0.08-0.2 inches (and all values and ranges therebetween), and the maximum inner diameter of the cavity through the longitudinal length of the expandable sheath after the expandable sheath being expanded can be at least 0.23 inches. The wall thickness of the expandable sheath can be generally less than 0.8 mm, and typically less than 0.5 mm. The longitudinal length of the expandable sheath can be at least 3 inches and typically 3-40 inches (and all values and ranges therebetween).

The shape of the expandable sheath 100 can optionally be tubular shaped or a cylindrical tube. The shape and size of the expandable sheath can be uniform along a majority (e.g., 6-99.99% and all values and ranges therebetween) or the full longitudinal length of the expandable sheath.

The expandable sheath 100 is configured to have a flexibility to allow the expandable sheath 100 to be bent along the longitudinal axis of the expandable sheath 100 so as to facilitate in the insertion of the expandable sheath 100 into a body passageway.

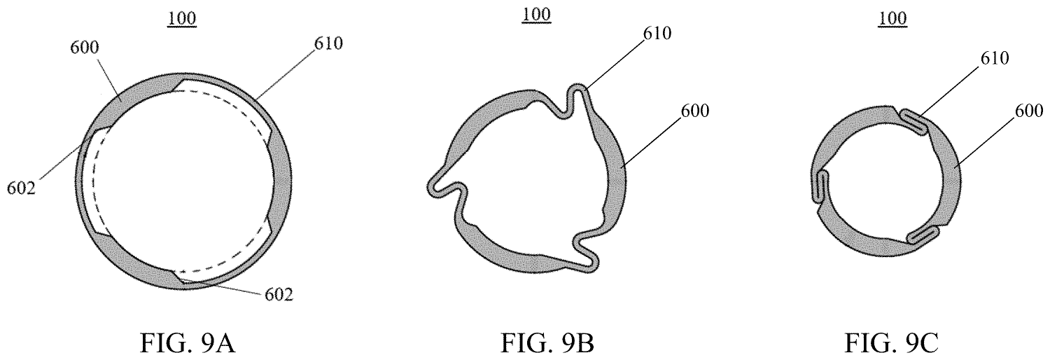

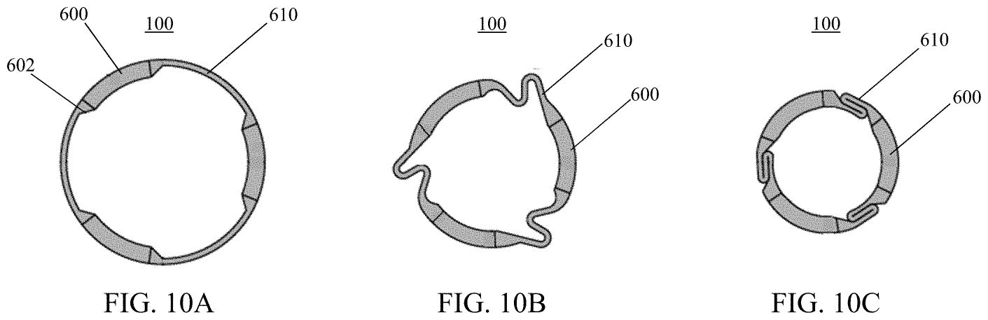

Referring now to FIGS. 9A-9C and 10A-10C, two other non-limiting embodiments of the expandable sheath 100 are illustrated. FIGS. 9A-9C and 10A-10C illustrate front end view or front face of the expandable sheath 100. The shape of front face of the front-end view of the expandable sheath 100 extends the full longitudinal length of the expandable sheath 100. The longitudinal length of the expandable sheath can be at least 3 inches and typically 3-40 inches (and all values and ranges therebetween). As such, the general side profile of the expandable sheath 100 can be the same or similar to the side profile of the expandable sheath 100 that is illustrated in in FIG. 2.

The expandable sheath 100 can have a wide variety of inner and outer diameters. The expandable sheath 100 can be configured to expand to an expanded outer diameter that is from about 10% greater than the original unexpanded outer diameter to about 300% greater than the original unexpanded outer diameter.

The expandable sheath 100 illustrated in FIGS. 9A-9C and 10A-10C can formed of a uniform material, and generally formed of a single material composition; however, it can be appreciated that the expandable sheath 100 can be formed of multiple materials.

The expandable sheath 100 illustrated in FIGS. 9A-9C and 10A-10C can formed of a single piece of material as illustrated in FIGS. 9A-9C, or can be formed of multiple pieces of material that are connected together as illustrated in FIGS. 10A-10C.

In one non-limiting embodiment, the expandable sheath illustrated in FIGS. 9A-9C and 10A-10C are formed of a single polymer or a single polymer mixture. In one specific non-limiting embodiment, the expandable sheath illustrated in FIGS. 9A-9C and 10A-10C are formed of styrenic block copolymer (SBC) or high-density polyethylene (HDPE). As can be appreciated, other polymers can be used.

In another non-limiting embodiment, the expandable sheath illustrated in FIGS. 9A-9C and 10A-10C are formed of a metal or metal alloy. In one specific non-limiting embodiment, the expandable sheath illustrated in FIGS. 9A-9C and 10A-10C are formed of shape memory metal alloy.

In another non-limiting embodiment, the expandable sheath illustrated in 10A-10C is formed of two different polymers or two different polymer mixtures. In one specific non-limiting embodiment, the thick bands 600 of the expandable sheath illustrated in 10A-10C are formed of high-density polyethylene (HDPE) and the thin bands 610 are formed of styrenic block copolymer (SBC). As can be appreciated, other polymers can be used.

In another non-limiting embodiment, the expandable sheath illustrated in FIGS. 9A-9C and 10A-10C are formed of one or more metals and/or metal alloys. In one specific non-limiting embodiment, the thick bands 600 of the expandable sheath illustrated in 10A-10C are formed of a metal or metal alloy and the thin bands 610 are formed of a shape memory material (e.g., a) copper-aluminum-nickel alloy, b) nickel-titanium alloy, c) zinc-copper-gold-iron alloy, d) Fe—Mn—Si alloy, e) Cu—Zn—Al alloy, f) Cu—Al—Ni alloy, g) Ag—Cd alloy, h) Au—Cd alloy, i) Co—Ni—Al alloy, j) Co—Ni—Ga alloy, k) Cu—Al—Be—X (X═Zr, B, Cr, Gd) alloy, l) Cu—Al—Ni—Hf alloy, m) Cu—Sn alloy, n) Cu—Zn alloy, o) Cu—Zn—X (X═Si, Al, Sn) alloy, p) Fe—Pt alloy, q) Mn—Cu alloy, r) Ni—Fe—Ga alloy, s) Ni—Ti—Hf alloy, t) Ni—Ti—Pd alloy, u) Ni—Mn—Ga alloy, v) Ni—Mn—Ga—Cu alloy, w) Ni—Mn—Ga—Co alloy, x) Ti—Nb alloy, etc.).

In another non-limiting embodiment, the expandable sheath illustrated in FIGS. 9A-9C and 10A-10C are formed of one or more metals and/or metal alloys, and one or more polymers. In one specific non-limiting embodiment, the thick bands 600 of the expandable sheath illustrated in 10A-10C are formed of a metal or metal alloy or a polymer (e.g., high-density polyethylene (HDPE), etc.) and the thin bands 610 are formed of a shape memory material (e.g., a) copper-aluminum-nickel alloy, b) nickel-titanium alloy, c) zinc-copper-gold-iron alloy, d) Fe—Mn—Si alloy, e) Cu—Zn—Al alloy, f) Cu—Al—Ni alloy, g) Ag—Cd alloy, h) Au—Cd alloy, i) Co—Ni—Al alloy, j) Co—Ni—Ga alloy, k) Cu—Al—Be—X (X═Zr, B, Cr, Gd) alloy, l) Cu—Al—Ni—Hf alloy, m) Cu—Sn alloy, n) Cu—Zn alloy, o) Cu—Zn—X (X═Si, Al, Sn) alloy, p) Fe—Pt alloy, q) Mn—Cu alloy, r) Ni—Fe—Ga alloy, s) Ni—Ti—Hf alloy, t) Ni—Ti—Pd alloy, u) Ni—Mn—Ga alloy, v) Ni—Mn—Ga—Cu alloy, w) Ni—Mn—Ga—Co alloy, x) Ti—Nb alloy, y) polyacrylate-based SMPs (e.g., t-butylacrylate-co-poly(ethyleneglycol) dimethacrylate (tBA-co-PEGDMA) polymers, etc.), z) (meth)acrylate-based SMPs, aa) polyurethane-based SMPs, and/or bb) blends of polyurethane and polyvinylchloride-based SMPs, etc.).

As illustrated in FIGS. 10A-10C, the thick bands 600 are formed of one piece of material and the thin bands 610 are formed of another piece of material and wherein the thick band 600 and thin band are connected together (e.g., adhesive connection, melted seam connection, etc.). In such an arrangement, the material used to form the thin band 610 can be the same or different from the material used to form the thick band 600.

In another non-limiting embodiment, the expandable sheath 100 illustrated in FIGS. 9A-9C and 10A-10C can be initially formed by an extrusion process. In one non-limiting process, the expandable sheath is formed by an extruded and the shape of the extruded expandable sheath is in the fully expanded orientation as illustrated in FIGS. 9A and 10A.

FIGS. 9A and 10A illustrate the expandable sheath 100 in the fully expanded orientation. FIGS. 9B and 10B illustrate the expandable sheath 100 in the partially folded configuration. FIGS. 9C and 10C illustrate the expandable sheath 100 in the fully folded position or fully contracted orientation.

When the expandable sheath 100 is in the fully contracted orientation, the diameter of the inner cavity is about 0.1-0.25 inches (and all values and ranges therebetween, 0.131 inches, etc.) and the outer diameter is about 0.105-0.27 inches (and all values and ranges therebetween, 0.171 inches, etc.). When the expandable sheath is in the fully expanded orientation, the diameter of the inner cavity is about 0.15-0.3 inches (and all values and ranges therebetween, 0.206 inches, etc.) and the outer diameter is about 0.155-0.35 inches (and all values and ranges therebetween, 0.246 inches, etc.). As can be appreciated, these dimensions are non-limiting and are only representative of a single non-limiting embodiment of the invention.

As illustrated in FIGS. 9 and 10, the thick bands 600 are generally not foldable or are non-foldable so that a portion of the thick band 600 folds over and overlaps another portion of the thick band 600 when the expandable sheath 100 moves between the fully expanded orientation and the fully contracted orientation. In one non-limiting arrangement, one, a plurality or all of the thick bands 600 maintain the same or substantially the same shape along the longitudinal axis of the expandable sheath 100 as the expandable sheath 100 moves between the fully expanded orientation and the fully contracted orientation. As also illustrated in FIGS. 9 and 10, the thin bands 610 are foldable. The foldability of the thin bands 610 can be at least partially the result of 1) the thin bands 610 are formed of a different material and/or a different number of layers from the thick bands 600, b) the thin bands 610 have a smaller thickness than the thick bands 600, c) the thin bands 610 have a different shape from the thick bands 600, and/or d) the thin bands 610 are processed differently (e.g., different heat treatments, different extrusion parameters, etc.) from the thick bands 600. Although the thick bands 600 have a rigidity that prevents the thick band form folding over itself as the expandable sheath 100 moves between the fully expanded orientation and the fully contracted orientation, both the thick bands 600 and thin bands 610 have a flexibility to allow the expandable sheath to be bent along the longitudinal axis of the expandable sheath 100 so as to facilitate in the insertion of the expandable sheath 100 into a body passageway.

As illustrated in FIGS. 9A-9C and 10A-10C, the expandable sheath 100 is illustrated as including three thin bands 610 and three thick bands 600. Generally, the expandable sheath 100 includes a plurality of thin bands 610 (e.g., 2-20 and all values and ranges therebetween) and a plurality of thick bands 600 (e.g., 2-20 and all values and ranges therebetween). Generally, two or more or all of the thin bands 610 have the same size, shape, configuration, and/or composition; however, this is not required. Generally, two or more or all of the thick bands 600 have the same size, shape, configuration, and/or composition; however, this is not required. As illustrated in FIGS. 9A-9C and 10A-10C, the size, shape and configuration of all the thin bands 610 are the same, and the size, shape and configuration of all the thick bands 600 are the same. The thickness of the thick band 610 is illustrated as being about 0.02-0.06 inches (and all values and ranges therebetween, 0.04 inches, etc.) thick; however, other thickness can be used. Generally, the thickness of the thin band 610 is generally about 10-80% (and all values and ranges therebetween) of the thickness of the thick band 600. Generally, the thickness of the thin band 610 is 30-50% the thickness of the thick band 600. Each end of a thick band 600 is connected to or merges with an end of an adjacently positioned thin band 610.

The total volume of the thick bands 600 is about 30-80 vol. % (and all values and ranges therebetween) of the expandable sheath. In one non-limiting embodiment, the total volume of the thick bands 600 is about 55-75 vol. % of the expandable sheath.

The total outer surface of the thick bands 600 constitutes about 30-80% (and all values and ranges therebetween) of the outer circumference of the expandable sheath when the expandable sheath is in the fully expanded orientation. In one non-limiting embodiment, total outer surface of the thick bands 600 constitutes about 45-65% of the outer circumference of the expandable sheath when the expandable sheath is in the fully expanded orientation.

As illustrated in FIGS. 9A-9C, the two interior side surfaces of each of the thin bands 610 are illustrated as having a tapered region 602 to transition to the thickness of the thick bands 600; however, this is not required. As illustrated in FIGS. 9 and 10, the thickness of the tapered region 602 is thickest at the point where the tapered region 602 terminates at the thick band 600.

Each of the tapered regions 602 are illustrated to have the same size, shape and configuration; however, this is not required. The length of each taper regions 602, when used, is generally 2-40% (and all values and ranges therebetween) of the circumferential length of the thin band 610. The thickness of each of the thin bands 610 is illustrated as being generally uniform along 80-100% (and all values and ranges therebetween) of the circumferential length of the thin band; however, this is not required.

As illustrated in FIGS. 10A-10C, the thin bands 610 may or may not be formed of a different material from the thick bands 600. Similar to the tapered regions 602 illustrated in FIGS. 9a-9C, the tapered regions 602 illustrated in FIGS. 10A-10C are located on the ends of the thin bands 610. Although the tapered regions 602 illustrated in FIGS. 9 and 10 are located in the thin portions 610, it can be appreciated that the thick bands 600 can alternatively include the tapered regions 602.

As illustrated in FIGS. 9A-9C and 10A-10C, the outer cross-sectional profile of the expandable sheath 100 in the fully expanded orientation and/or the fully contracted orientation is generally circular. The cross-sectional shape of the internal cavity of the expandable sheath 100 in the fully contracted orientation is generally circular. The cross-sectional shape of the internal cavity of the expandable sheath 100 in the fully expanded orientation is also generally circular, except for the recessed portions formed by the tapered regions on the thick bands 600 and the thinner thickness of the thin bands 610. As illustrated in FIGS. 9A-9C and 10A-10C, the three recessed portions are equally spaced from adjacently positioned recessed positions about the interior cavity circumference of the expandable sheath 100 when the expandable sheath 100 is in the fully expanded orientation. Generally, the recessed portions formed by the thin bands 610 are equally spaced about the circumference of the expandable sheath 100; however, this is not required. The shape, size and/or configuration of the recessed portions can be the same; however, this is not required.

After the expandable sheath 100 is formed in the fully expanded orientation (e.g., formed by extrusion, co-extrusion, stamping, molding, etching, etc.), the expandable sheath 100 can be formed into the final fully contracted orientation by causing the thin bands 610 to be bent or folded and/or optionally twisted. The arrangement used to bend or fold and/or optionally twist the thin bands 610 is non-limiting. As illustrated in FIGS. 9C and 10C, the thin bands 610 are bent at about one third the circumferential length of the thin bands 610 to form a bent thin band 610 having three stacked layers. As can be appreciated, the thin band 610 can be bent to form more than three stacked layers (e.g., 5 layers, 7 layers, etc.). The length of each of the overlapping layers of the one or more thin bands 610 can be the same or substantially the same as illustrated in FIGS. 9C and 10C; however, this is not required. As illustrated in FIGS. 9C and 10C, the thin bands 610 have a generally S-shaped or serpentine or sinuous configuration when the expandable sheath 100 in the fully contracted orientation. When the expandable sheath 100 is expanded to the fully expanded orientation as illustrated in FIGS. 9A and 10A, the one or more thin bands 610 are caused to be reshaped from the S-shape or serpentine or sinuous configuration to an arc-shape configuration. In the fully expanded configuration, one or more of the thin bans 610 do not have any overlapping portion as illustrated in FIGS. 9A and 10A.

As illustrated in FIGS. 9C and 10C, the two rounded ends of the three stack or overlapping layers of the thin bands 610 are being positioned adjacent to a tapered region 602 of a thin band 610. As illustrated in FIGS. 9C and 10C, the folded thin bands are equally displaced from each other around the circumference of the expandable sheath 100; however, this is not required.

Generally, the thin bands 610 are partially or fully formed of a shape memory material. The thick bands may or may not be partially or fully formed of a shape memory material.

The thin bands 610 can optionally be folded prior to being fully cooled (when the polymer is heated during the extrusion of the one or more polymers when forming the expandable sheath) and/or the folded thin bands 610 can be optionally heated and then cooled while in a folded state so as to create some memory in the thin bands 610 so that the thin bands 610 want to maintain its folded or partially folded state after the expandable sheath 100 has been expanded from its fully contracted orientation. The heat treatment process may vary when the shape memory material is a polymer or a metal alloy. Such a shape memory feature can be advantageous 1) maintaining the medical device in a non-expanded orientation during the deployment of a medical device in a patient; 2) causing the expandable sheath 100 to partially or fully return to its fully contracted orientation after expansion be a medical device being passed through the expandable sheath 100; and/or 3) causing the expandable sheath 100 to partially or fully return to its fully contracted orientation to facilitate in the removal of the expandable sheath 100 from a patient after the medical device has been inserted into the patient.

One or more outer surfaces of the thin bands 610 and/or thick bands 600 can optionally include a lubrication coating and/or liner to facilitate in the movement of a medical device through the expandable sheath 100. In one non-limiting embodiment, the surfaces of the thin bands 610 and/or thick bands 600 that are coated with a lubrication coating and/or includes a liner has a coefficient of friction of no more about 0.1 (e.g., 0.0001-0.1 and all values and ranges therebetween). Non-limiting examples of lubrication materials include PTFE, polyethylene, polyvinylidene fluoride, and combinations thereof In one non-limiting configuration, the inner surface of the inner layer portion includes a coating of PTFE.

One or more outer surfaces of the thin bands 610 and/or thick bands 600 can optionally include a hydrophilic coating to facilitate in the insertion of the expandable sheath 100 into and/or out of a body passageway. Non-limiting examples of hydrophilic coatings include the Harmony™ Advanced Lubricity Coatings and other Advanced Hydrophilic Coatings available from SurModics, Inc., Eden Prairie, Minn. DSM medical coatings (available from Koninklijke DSM N.V, Heerlen, the Netherlands).

The expandable sheath 100 can optionally include one or more radiopaque markers or fillers. The radiopaque filler or marker, when used, can be located on the outer surface of the expandable sheath, embedded on one or more layers of the expandable sheath, and/or be located between on or more layers of the expandable sheath. The location of the one or more radiopaque markers or fillers on the expandable sheath 100 is non-limiting. Non-limiting materials that can be used as a radiopaque filler or marker include barium sulfite, bismuth trioxide, titanium dioxide, and/or bismuth subcarbonate.

The expandable sheath 100 in accordance with the present disclosure can be used a) to minimize trauma to a body passageway (e.g., blood vessel, etc.) by allowing for temporary expansion of a portion of the expandable sheath to accommodate a medical device and/or a delivery system for a medical device, and thereafter the expandable sheath is configured to return to its original diameter or close to its original diameter once the medical device and/or a portion of the delivery system passes through expandable sheath 100, b) to reduce the length of time a procedure takes, c) to reduce the risk of a longitudinal or radial body passageway tear, d) to reduce risk of plaque dislodgement in a body passageway, e) to reduce or eliminate the need for multiple insertions sheaths or other devices for the dilation of a body passageway, f) for many types of minimally invasive surgery, such as any surgery requiring introduction of a medical device (e.g., stent, prosthetic heart valve, grafts, etc.) into a body passageway (e.g., veins, arteries, esophagus, ducts of the biliary tree, intestine, urethra, fallopian tube, other endocrine or exocrine ducts, etc.).

The expandable sheath 100, in accordance with the present disclosure, can be used with various methods of introducing a medical device into a patient's vasculature. One such method comprises positioning an expandable sheath in a patient's vessel, passing a medical device through the expandable sheath 100, which causes a portion of the expandable sheath surrounding the device to expand and accommodate the profile of the medical device, and then retracting the expanded portion of the expandable sheath 100 to its original size or near original size after the medical device has partially or fully passed through the expandable sheath 100. In some methods, the expandable sheath 100 can be sutured to the patient's skin at the insertion site so that once the expandable sheath 100 is inserted the proper distance within the patient's vasculature, it does not move once the implantable medical device starts to travel through the expandable sheath 100.

The expandable sheath 100 in accordance with the present disclosure can be used with other delivery and minimally invasive surgical components, such as an introducer and loader. In one non-limiting embodiment, the expandable sheath 100 can be flushed to purge any air within the expandable sheath 100. An introducer can be inserted into the expandable sheath 100 and the introducer/sheath combination can be fully inserted into vasculature over a guiding device, such as a guidewire. Once the expandable sheath 100 and introducer are fully inserted into a patient's vasculature, the expandable sheath 100 can be optionally sutured in place at the insertion site. In this manner, the expandable sheath 100 can be substantially prevented from moving once positioned within the patient. The introducer can then be removed and a medical device, such as a transcatheter heart valve can be inserted through the expandable sheath 100. Such methods can additionally comprise placing the heart valve in a crimped state on the distal end portion of an elongated delivery apparatus, and inserting the elongated delivery device with the crimped valve into and through the expandable sheath 100. Next, the delivery apparatus can be advanced through the patient's vasculature to the treatment site, where the valve can be implanted. Typically, the medical device has a greater outer diameter than the diameter of the expandable sheath 100 in its fully contracted orientation. The medical device can be advanced through the expandable sheath 100 towards the implantation site, and the expandable sheath 100 can locally expand to accommodate the medical device as the device passes through the expandable sheath 100. The radial force exerted by the medical device on the interior passageway or cavity of the expandable sheath 100 can be sufficient to locally expand the expandable sheath 100 to an expanded diameter (e.g., the fully expanded orientation) just in the area where the medical device is currently located or the complete sheath can be caused to be expanded along its longitudinal length. Once the medical device passes a particular location of the expandable sheath 100, the expandable sheath 100 can at least partially contract to a smaller diameter. Alternatively, the expandable sheath 100 can be configured to contract after the medical device has fully passed through the expandable sheath 100. Once the medical device is implanted, the expandable sheath 100 and any sutures holding it in place can be removed.

Although example embodiments have been disclosed, a worker of ordinary skill in this art would recognize that certain modifications would come within the scope of the claims. For that reason, the following claims should be studied to determine their true scope and content.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure which, as a matter of language, might be said to fall therebetween. The disclosure has been described with reference to the preferred embodiments. These and other modifications of the preferred embodiments, as well as other embodiments of the disclosure, will be obvious from the disclosure herein, whereby the foregoing descriptive matter is to be interpreted merely as illustrative of the disclosure and not as a limitation. It is intended to include all such modifications and alterations insofar as they come within the scope of the appended claims.

To aid the Patent Office and any readers of this application and any resulting patent in interpreting the claims appended hereto, applicants do not intend any of the appended claims or claim elements to invoke 35 U.S.C. 112(f) unless the words “means for” or “step for” are explicitly used in the particular claim.

What is claimed:

1. An expandable sheath that provides access to a body passageway for delivering a medical device into the body passageway; said expandable sheath expandable from a first diameter to a second diameter; said second diameter greater than said first diameter; said expandable sheath including an internal cavity that extends a longitudinal length of said expandable sheath; said expandable sheath includes a plurality of thin bands and plurality of thick bands that extend along a longitudinal length of said expandable sheath; each of said thin bands include first and second ends; each of said thick bands includes first and second ends; said first end of said thick bands and thin bands are connected together; said second end of said thick bands and thin bands are connected together; at least one of said thin bands configured to increase in longitudinal length when said expandable sheath expands from said first diameter to said second diameter; and
wherein said expandable sheath is configured to expand to said second diameter during movement of the medical device through said internal cavity of said expandable sheath; said expandable device configured to contract from said second diameter to said first diameter after the medial device partially or fully passes through said internal cavity of said expandable sheath.

2. The expandable sheath as defined in claim 1, wherein one or more of said thin bands is partially or fully formed of a shape memory material.

3. The expandable sheath as defined in claim 1, wherein one or more of said thin bands is formed of a different material from one or more of said thick bands.

4. The expandable sheath as defined in claim 1, wherein a) one or more of said thin bands has an S-shaped configuration when said expandable sheath in said first diameter, and wherein one or more of said thin bands is reshaped from said S-shape configuration to an arc-shape when said expandable sheath expands from said firsts diameter to said second diameter, or b) one or more of said thin bands have overlapping portions when said expandable sheath in said first diameter, and wherein one or more of said thin bands is reshaped from being in an overlapping configuration to a non-overlapping configuration when said expandable sheath expands from said firsts diameter to said second diameter.

5. The expandable sheath as defined in claim 1, including a tapered transition having a variable thickness located at end portion of one or more of said thin bands and terminates at said first or second end of said thick band.

6. The expandable sheath as defined in claim 1, wherein a circumferential length of said one or more of said thin bands is equal to or greater than a circumferential length of said one or more of said thick bands when said expandable sheath is in said second diameter.

7. The expandable sheath as defined in claim 1, wherein said thin bands and/or said thick bands is partially or fully formed of a polymer material.

8. The expandable sheath as defined in claim 1, wherein said thin bands and/or said thick bands is partially or fully formed of a metal material.

9. The expandable sheath as defined in claim 1, wherein a) a plurality or all of said thick bands have the same shape, size, configuration and/or composition, and/or b) a plurality or all of said thin bands have the same shape, size, configuration and/or composition.

10. The expandable sheath as defined in claim 1, wherein a thinnest thickness of one or more said thin bands is 10-80% of a thickness of a maximum thickness of one or more of said thick bands.

11. A method of introducing a prosthetic heart valve into a patient's vasculature, said method comprising:
inserting an expandable sheath at least partially into a blood vessel of a patient; said expandable sheath expandable from a first diameter to a second diameter; said second diameter greater than said first diameter; said expandable sheath including an internal cavity that extends a longitudinal length of said expandable sheath; said expandable sheath includes a plurality of thin bands and plurality of thick bands that extend along a longitudinal length of said expandable sheath; each of said thin bands include first and second ends; each of said thick bands includes first and second ends; said first end of said thick bands and thin bands are connected together; said second end of said thick bands and thin bands are connected together; at least one of said thin bands configured to increase in longitudinal length when said expandable sheath expands from said first diameter to said second diameter; and
advancing a prosthetic heart valve through said internal cavity of said expandable sheath so as to cause said expandable sheath to expand from said first diameter to said second diameter as said prosthetic heart valve partially or fully passes through said internal cavity of said expandable sheath; and
wherein said expandable sheath contracts from said second diameter to said first diameter after said prosthetic heart valve has partially or fully passed through said internal cavity of said expandable sheath.

12. The method as defined in claim 11, wherein said prosthetic heart valve is a stent mounted heart valve mounted in a radially crimped state on a delivery apparatus.

13. A sheath adapted for use in the delivery of a medical device into a body passageway; said sheath comprising:
an outer layer portion;
an inner layer portion; and
a frame; said frame at least partially positioned between said inner and outer layer portions; and
wherein said sheath is configured to expand from a first outer diameter to a second outer diameter as the medical device is moved through said sheath, and thereafter said sheath partially or fully returns to said first outer diameter once the medical device has passed partially or fully through said sheath;
wherein said sheath include an internal cavity extending said longitudinal length of said sheath; and
wherein said sheath having a longitudinal length.

14. The sheath as defined in claim 13, wherein said outer layer portion includes a polymer material.

15. The sheath as defined in claim 14, wherein said outer layer portion includes a first and second polymer layer.

16. The sheath as defined in claim 13, wherein a thickness of said outer layer portion is greater than a thickness of said inner layer portion.

17. The sheath as defined in claim 13, wherein said inner layer portion includes a polymer material.

18. The sheath as defined in claim 17, wherein said inner layer portion includes a first and second polymer layer.

19. The sheath as defined in claim 13, wherein said frame includes a shape memory material.

20. The sheath as defined in claim 19, wherein said shape memory material includes a nickel-titanium alloy.

21. The sheath as defined in claim 13, wherein said frame is at least partially positioned between said inner and outer layer portions.

22. The sheath as defined in claim 13, wherein said frame includes first and second frame wires that extend along a majority of said longitudinal length of said sheath; a majority of a longitudinal length of each of said first and second frame wires do not overlap one another along a majority of said longitudinal length of said sheath.

23. The sheath as defined in claims 13, wherein said first and second frame wires include a plurality of undulations along a majority of said longitudinal length of each of said first and second frame wires; a majority of a longitudinal length of each of said first and second frame wires do not overlap one another along a majority of said longitudinal length of said sheath.

24. The sheath as defined in claim 13, further including longitudinal shaping structure;
said longitudinal shaping structure configured to inhibit kinking of said sheath.

25. The sheath as defined in claim 24, wherein said longitudinal shaping structure is at least partially located in said outer layer portion.

26. The sheath as defined in claim 24, wherein said longitudinal shaping structure is positioned along a majority of said longitudinal length of said sheath.

27. The sheath as defined in claim 24, wherein said longitudinal shaping structure include shape memory material.

28. The sheath as defined in claim 13, wherein a) a first end of said sheath is subjected to a reflow process to connect together said inner and outer layer portions; a majority of said longitudinal length of said sheath not subjected to said reflow process, and/or b) a second end of said sheath is subjected to a reflow process to connect together said inner and outer layer portions;
a majority of said longitudinal length of said sheath not subjected to said reflow process.

29. A method of introducing a medical device into a body passageway of a patient; said method comprising:
a. providing an expandable sheath; said sheath comprising a) an outer layer portion, b) an inner layer portion, and c) a frame; said frame at least partially positioned between said inner and outer layer portions; and wherein said sheath is configured to expand from a first outer diameter to a second outer diameter as the medical device is moved through said sheath, and thereafter said sheath partially or fully returns to said first outer diameter once the medical device has passed partially or fully through said sheath;
b. inserting said expandable sheath at least partially into said body passageway;
c. advancing said medical device through said expandable sheath to cause said expandable sheath to locally expand from said first outer diameter to said second outer diameter due to movement of said medical device through said expandable sheath; and
wherein said expandable sheath contracts back to said first outer diameter after said medical device as partially or fully passed through said expandable sheath.

30. The method as defined in claim 29, wherein said step of inserting includes passing said expandable sheath transcutaneously through a surgically-created opening in the patient's skin such that at least a portion of said outer portion of said expandable sheath is positioned adjacent to said surgically-created opening.

31. The method as defined in claim 29, wherein said medical device in a stent mounted heart valve mounted in a radially crimped state on a delivery apparatus.
